(19) United States
(12) Patent Application Publication
(10) Pub. No.: US 2026/0041765 A1
(43) Pub. Date: Feb. 12, 2026

(54) PHARMACEUTICAL COMPOSITION OF PVRIG/TIGIT BISPECIFIC ANTIBODY AND USE THEREOF

(71) Applicant: SHANDONG SIMCERE BIOPHARMACEUTICAL CO., LTD., Shandong (CN)

(72) Inventors: Xing CHEN, Shanghai (CN); Xinxin LI, Shanghai (CN); Jianzhong HU, Shanghai (CN); Ande LUO, Shanghai (CN); Xiaofeng ZHAO, Shanghai (CN); Renhong TANG, Shanghai (CN)

(21) Appl. No.: 18/870,173

(22) PCT Filed: Jun. 7, 2023
(86) PCT No.: PCT/CN2023/098806

§ 371 Date: Nov. 27, 2024

(30) Foreign Application Priority Data
Jun. 8, 2022 (CN) 202210641617.6

(51) Int. Cl. A61K 39/395; A61K 47/12; A61K 47/26; A61P 35/00; C07K 16/28

(52) U.S. Cl. CPC A61K 39/39558; A61K 47/12; A61K 47/26; A61P 35/00; C07K 16/28; C07K 16/2896; C07K 2317/24; C07K 2317/31; C07K 2317/569; C07K 2317/73; C07K 2317/92

(57) ABSTRACT

Provided are a pharmaceutical composition of a PVRIG/TIGIT bispecific antibody and use thereof. Specifically, provided is a pharmaceutical composition, comprising a PVRIG/TIGIT bispecific antibody and a buffer solution. The pharmaceutical composition further comprises a sugar and a surfactant. Also provided is a pharmaceutical composition for treating cancer or infectious diseases.

The present application claims priority to the Chinese Patent Application No. 202210641617.6 entitled “PHARMACEUTICAL COMPOSITION OF PVRIG/TIGIT BISPECIFIC ANTIBODY AND USE THEREOF”, filed with the China National Intellectual Property Administration on Jun. 8, 2022, which is incorporated herein by reference in its entirety.

### TECHNICAL FIELD

The present disclosure relates to the field of pharmaceutical formulations, in particular to a pharmaceutical composition comprising a PVRIG/TIGIT bispecific antibody or an antigen-binding fragment thereof.

### BACKGROUND

Immunotherapy, which is based on the manipulation and/or modulation of the immune system, including both innate and acquired immune responses, generally aims to treat diseases by controlling the immune response to “foreign agents” (e.g., pathogens or tumor cells). The immune system is a highly complex system composed of numerous cell types with complex and subtle systems that control those interactions and reactions. The concept of cancer immunosurveillance is based on the theory that the immune system can recognize tumor cells, initiate an immune response, and inhibit tumor development and/or progression. However, it is clear that many cancer cells have developed mechanisms to evade the immune system, allowing for uninhibited tumor growth. Cancer/Tumor immunotherapy has focused on the development of new and novel agonists and/or antagonists that can activate and/or excite the immune system to achieve a more effective anti-tumor response, enhance the killing of tumor cells, and/or inhibit tumor growth.

In recent years, immune checkpoint therapy against immune cell co-inhibitory receptors has made great progress in tumor immunotherapy, and the discovery and validation of new co-inhibitory receptors has become a global competitive hotspot. PVRIG is expressed in NK cells and T cells and shares several similarities with other known immune checkpoints. When PVRIG binds to its ligand (PVRL2), it elicits an inhibitory signal that acts to attenuate the immune response of NK cells and T cells against target cells (i.e., similar to PD-1/PD-L1). Blocking the binding of PVRL2 to PVRIG will cut off this inhibitory signal from PVRIG and thereby modulate the immune response of NK cells and T cells. Similarly, TIGIT is another target of interest, and it has been demonstrated that its binding to its cognate ligand PVR directly inhibits the cytotoxicity of NK cells and T cells through its intracellular ITIM domain. Knockout of the TIGIT gene or blocking antibodies to the TIGIT/PVR interaction have been shown to enhance NK cell killing in vitro or exacerbate autoimmune diseases in vivo.

TIGIT and PVRIG belong to DNAM superfamily, and are proved to be co-expressed in a plurality of tumor-infiltrating lymphocytes to play an immunosuppressive role. Bispecific antibodies capable of targeting both PVRIG and TIGIT have a potential synergistic effect and are an attractive treatment mode for single antibody therapy. Antibodies are proteins with relatively high molecular weight. Due to the instability in solutions, proteins are very prone to forming particles and aggregates, thereby making stability a challenge in the development of large-molecule protein drugs. Therefore, there is a need for stable formulations of PVRIG/TIGIT bispecific antibodies for pharmaceutical use, e.g., for the treatment of various cancers and infectious diseases. Such formulations have good stability at room temperature or high temperatures, which enables a reduction in the cost of transporting and storing antibody drugs.

### SUMMARY

The present disclosure discloses a pharmaceutical composition of a PVRIG/TIGIT bispecific antibody and use thereof.

In one aspect, the present disclosure provides a pharmaceutical composition comprising:
(i) a bispecific antibody or an antigen-binding fragment thereof comprising a binding domain that specifically binds to PVRIG and TIGIT,
(ii) a buffer,
(iii) a non-reducing sugar, and
(iv) a non-ionic surfactant, wherein the bispecific antibody or the antigen-binding fragment thereof comprises:
(a) a first antigen-binding moiety comprising a heavy chain variable region (VH) and a light chain variable region (VL), where the VH and VL form an anti-TIGIT antigen-binding domain, where the TIGIT VH comprises HCDR1, HCDR2, and HCDR3 of the VH set forth in any one of SEQ ID NO: 12 or 14; the TIGIT VL comprises LCDR1, LCDR2, and LCDR3 of the VL set forth in any one of SEQ ID NO: 11 or 13; and
(b) a second antigen-binding moiety comprising a VHH that specifically binds to PVRIG, where the VHH comprises CDR1, CDR2, and CDR3 set forth in any one of SEQ ID NO: 9 or 10.

Preferably, the first antigen-binding moiety comprises HCDR1, HCDR2, HCDR3, LCDR1, LCDR2, and LCDR3 of the following sequences:
(1) SEQ ID NOs: 21, 22, 23, 27, 28, and 29, respectively; or
(2) SEQ ID NOs: 24, 25, 26, 30, 31, and 32, respectively; or
(3) sequences having at least 90% identity or having 1, 2, 3, or more amino acid insertions, deletions, and/or substitutions compared with the sequences set forth in (1) to (2) described above, wherein preferably, the substitutions are conservative amino acid substitutions;
the second antigen-binding moiety comprises CDR1, CDR2, and CDR3 of the following sequences:
(1) SEQ ID NOs: 15, 16, and 17, respectively; or
(2) SEQ ID NOs: 18, 19, and 20, respectively; or
(3) sequences having at least 90% identity or having 1, 2, 3, or more amino acid insertions, deletions, and/or substitutions compared with the sequences set forth in (1) to (2) described above, wherein preferably, the substitutions are conservative amino acid substitutions.

Preferably, the VH of the first antigen-binding moiety comprises a sequence having at least 90% identity to the amino acid sequence set forth in SEQ ID NO: 12 or 14: the VL of the first antigen-binding moiety comprises a sequence having at least 90% identity to the amino acid sequence set forth in SEQ ID NO: 11 or 13: the second antigen-binding moiety comprises a sequence having at least 90% identity to the amino acid sequence set forth in SEQ ID NO: 9 or 10.

In some embodiments, provided is the pharmaceutical composition as described in any one of the foregoing, and the PVRIG/TIGIT bispecific antibody or the antigen-binding fragment thereof is at a concentration of about 10 mg/mL to about 200 mg/mL, preferably, about 10 mg/mL to about 150 mg/mL, about 15 mg/mL to about 120 mg/mL, about 20 mg/mL to about 100 mg/mL, about 20 mg/mL to about 80 mg/mL, about 20 mg/mL to about 70 mg/mL, or about 30 mg/mL to about 60 mg/mL.

In some embodiments, provided is the pharmaceutical composition as described in any one of the foregoing, and the buffer is selected from an acetic acid-sodium acetate buffer or a histidine-histidine hydrochloride buffer; preferably, the buffer is at a concentration of about 10 mM to about 80 mM, about 15 mM to about 70 mM, about 20 mM to about 60 mM, about 20 mM to about 50 mM, about 20 mM to about 40 mM, or about 20 mM to about 30 mM.

In some embodiments, provided is the pharmaceutical composition as described in any one of the foregoing, and the non-reducing sugar is selected from sucrose, sorbitol, or trehalose; preferably, the non-reducing sugar is at a concentration of about 6% to about 10% (w/v), about 6% to about 9% (w/v), about 7% to about 9% (w/v), or about 7% to about 8% (w/v).

In some embodiments, provided is the pharmaceutical composition as described in any one of the foregoing, and the non-ionic surfactant is polysorbate 80; preferably, the non-ionic surfactant is at a concentration of about 0.01% to about 0.10% (w/v), about 0.01% to about 0.08% (w/v), about 0.02% to about 0.07% (w/v), or about 0.02% to about 0.06% (w/v).

In some embodiments, the pharmaceutical composition as described in any one of the foregoing is an injection, preferably a subcutaneous injection, an intravenous injection, or an intravenous infusion.

In some embodiments, the pharmaceutical composition as described in any one of the foregoing comprises:
(i) about 10 mg/mL to about 200 mg/mL of the PVRIG/TIGIT bispecific antibody or the antigen-binding fragment thereof;
(ii) about 10 mM to about 100 mM acetic acid-sodium acetate buffer;
(iii) about 6% to about 10% (w/v) sucrose; and
(iv) about 0.01% to about 0.10% (w/v) polysorbate 80.

Preferably, the pharmaceutical composition comprises about 50 mg/ml of the PVRIG/TIGIT bispecific antibody or the antigen-binding fragment thereof, 20 mM acetic acid-sodium acetate buffer, about 8% w/v sucrose, and about 0.02% polysorbate 80.

Preferably, the pharmaceutical composition comprises about 50 mg/ml of the PVRIG/TIGIT bispecific antibody or the antigen-binding fragment thereof, 20 mM acetic acid-sodium acetate buffer, about 8% w/v sucrose, and about 0.04% polysorbate 80.

Preferably, the pharmaceutical composition comprises about 50 mg/ml of the PVRIG/TIGIT bispecific antibody or the antigen-binding fragment thereof, 20 mM acetic acid-sodium acetate buffer, about 8% w/v sucrose, and about 0.06% polysorbate 80.

In some embodiments, the pH of the pharmaceutical composition as described in any one of the foregoing is about 4.5-5.5, preferably, about 5.0-5.2.

In some embodiments, the pharmaceutical composition as described in any one of the foregoing is stable at 2° C., to 8° C. for at least 4 weeks, 5 weeks, 6 weeks, 7 weeks, 8 weeks, 3 months, or 6 months.

In some embodiments, the pharmaceutical composition as described in any one of the foregoing is stable at 25° C. for at least 4 weeks, 5 weeks, 6 weeks, 7 weeks, 8 weeks, 3 months, or 6 months.

In some embodiments, the pharmaceutical composition as described in any one of the foregoing is stable at 40° C. for at least 1 week, 2 weeks, 3 weeks, or 4 weeks.

In another aspect, provided is use of the pharmaceutical composition as described in any one of the foregoing for the manufacture of a medicament for treating a cancer or an infectious disease, where the cancer is selected from a solid tumor and a hematologic tumor.

In some embodiments, the pharmaceutical composition is used in combination with an additional therapeutic agent or surgery, where the additional therapeutic agent is selected from radiation therapy, chemotherapy, an oncolytic drug, a cytotoxic agent, a cytokine, an immunostimulatory antibody, an immunomodulatory drug, an activator of a costimulatory molecule, an inhibitor of an inhibitory molecule, a vaccine, or cellular immunotherapy, and the surgery is a surgical treatment.

In another aspect, the present disclosure provides a method for treating a cancer or an infectious disease, and the method comprises administering to a patient in need thereof an effective amount of the pharmaceutical composition as described in any one of the foregoing, where the cancer is selected from a solid tumor and a hematologic tumor.

In another aspect, the present disclosure provides the pharmaceutical composition as described in any one of the foregoing for use in treating a cancer or an infectious disease, where the cancer is selected from a solid tumor and a hematologic tumor.

### Terminology and Definitions

Unless otherwise stated, the terms used herein have the meanings that are commonly understood by those of ordinary skill in the art. For a term explicitly defined herein, the meaning of the term shall be subject to the definition.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “include”, “comprise”, and “have” herein are used interchangeably and are intended to indicate the inclusion of a solution, implying that there may be elements other than those listed in the solution. Meanwhile, it should be understood that the descriptions “include”, “comprise”, and “have” as used herein also provide the solution of “consist of . . . ”. Illustratively, “a composition, comprising A and B” should be understood as the following technical solution: a composition consisting of A and B, and a composition containing other components in addition to A and B, all fall within the scope of the aforementioned “a composition”.

The term “and/or” as used herein includes the meanings of “and”, “or”, and “all or any other combination of elements linked by the term”.

The “about” before a value as used herein means that the value can be varied up or down, with an optional variation of 10%. For example, “about 10%” refers to “9%-11%”: the “about” before a numerical range as used herein means that the endpoints of the numerical range can fluctuate up and down, with the upper endpoint fluctuating up and the lower endpoint fluctuating down, e.g., “about 10-20 nM” means a range of “9-22 nM”. Alternatively, the “about” may also range up to 5%, 4%, 2%, or 1% fluctuation.

The terms “T cell immunoreceptor with Ig and ITIM domains”, “TIGIT”, “TIGIT antigen”, “Vstm3”, and “WUCAM” are used interchangeably and include various mammalian isoforms, such as human TIGIT, orthologs of human TIGIT, and analogs comprising at least one epitope within TIGIT, as well as analogs having at least one epitope in common with TIGIT. The amino acid sequences of TIGIT (e.g., human TIGIT) and the nucleotide sequences encoding them are known in the art.

The term “PVRIG” or “PVRIG protein” herein can optionally include any such protein or variant, conjugate or fragment thereof, including, but not limited to, known or wild-type PVRIG as described herein, as well as any naturally occurring splice variant, amino acid variant, or isoform, and in particular ECD fragments of PVRIG. An “anti-PVRIG antibody” (including an antigen-binding fragment) that binds to PVRIG and prevents its activation by PVRL2 (e.g., most often by blocking the interaction of PVRIG and PVLR2) is used to enhance T cell and/or NK cell activation and to treat diseases such as cancer and pathogen infection.

The PVRIG/TIGIT bispecific antibody of the present disclosure specifically binds to human TIGIT, preferably the ECD of human TIGIT, and PVRIG more preferably the ECD of human PVRIG.

The term “specifically bind to” herein refers to that an antigen-binding molecule (e.g., an antibody) specifically binds to an antigen and substantially identical antigens, generally with high affinity, but does not bind to unrelated antigens with high affinity. The affinity is generally reflected in an equilibrium dissociation constant (KD), where a relatively low KD indicates a relatively high affinity. In the case of antibodies, high affinity generally means having a KD of about 1×10⁻⁷ M or less, about 1×10⁻⁸ M or less, about 1×10⁻⁹ M or less, about 1×10⁻¹⁰ M or less, 1×10⁻¹¹ M or less, or 1×10⁻¹² M or less. The KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis.

The term “antigen-binding molecule” herein is used in its broadest sense and refers to a molecule that specifically binds to an antigen. Illustratively, the antigen-binding molecule includes, but is not limited to, an antibody or an antibody mimetic. “Antibody mimetic” refers to an organic compound or a binding domain that is capable of specifically binding to an antigen, but is not structurally related to an antibody. Exemplarily, the antibody mimetic includes, but is not limited to, affibody, affitin, affilin, a designed ankyrin repeat protein (DARPin), a nucleic acid aptamer, and a Kunitz domain peptide.

The term “antibody” herein is used in its broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen-binding activity. The “antibody” herein includes alternative protein scaffolds or artificial scaffolds having grafted complementarity determining regions (CDRs) or CDR derivatives. Such scaffolds include antibody-derived scaffolds comprising mutations introduced to, for example, stabilize the three-dimensional structure of the antibody, and fully synthetic scaffolds comprising, for example, biocompatible polymers. See, e.g., Korndorfer et al., 2003, Proteins: Structure. Function, and Bioinformatics, 53 (1):121-129 (2003); and Roque et al., Biotechnol. Prog. 20:639-654 (2004). Such scaffolds may also include non-antibody derived scaffolds, such as scaffold proteins known in the art to be useful for grafting CDRs, including, but not limited to tenascin, fibronectin, peptide aptamers, and the like.

The “antibody” herein may be derived from any animal, including, but not limited to, human and non-human animals which may be selected from primates, mammals, rodents, and vertebrates, such as Camelidae species, Lama glama, Lama guanicoe, Vicugna pacos, sheep, rabbits, mice, rats, or Chondrichthyes species (e.g., shark).

“Antibody” herein includes but is not limited to, monoclonal antibodies, polyclonal antibodies, monospecific antibodies, multispecific antibodies (e.g., bispecific antibodies), monovalent antibodies, multivalent antibodies, intact antibodies, fragments of an intact antibody, naked antibodies, conjugated antibodies, chimeric antibodies, humanized antibodies, or fully human antibodies.

The term “monoclonal antibody” herein refers to an antibody obtained from a population of substantially homogeneous antibodies, that is, the individual antibodies constituting the population are identical and/or bind to the same epitope, except for possible variants (e.g., containing naturally occurring mutations or arising during the production of the formulation, such variants typically being present in minor amounts). In contrast to polyclonal antibody formulations, which generally comprise different antibodies directed against different determinants (epitopes), each monoclonal antibody in a monoclonal antibody formulation is directed against a single determinant on the antigen. The modifier “monoclonal” herein is not to be construed as requiring the production of the antibody or the antigen-binding molecule by any particular method. For example, monoclonal antibodies can be prepared by a variety of techniques, including (but not limited to) a hybridoma technique, a recombinant DNA method, a phage library display technique, methods that utilize transgenic animals containing all or part of human immunoglobulin loci, and other methods known in the art.

The terms “antigen-binding fragment” and “antibody fragment” herein are used interchangeably and refer to a fragment that does not have the entire structure of an intact antibody, but comprises only a portion of the intact antibody or a variant of the portion that retains the ability to bind to an antigen. “Antigen-binding fragment” or “antibody fragment” herein includes but is not limited to, a Fab, a Fab′, a Fab′-SH, a F(ab′)2, an Fd, an Fv, an scFv, a diabody, and a single domain antibody.

The term “multispecific” herein means having at least two antigen-binding sites, that is, at least “bispecific”, which may be “trispecific”, “tetraspecific”, etc.; each of which binds to a different epitope of the same antigen or a different epitope of a different antigen. In addition, “bispecific” can also be an antibody-derived molecule having at least two antigen-binding sites, such as an immunoconjugate.

The term “humanized antibody” herein refers to a genetically engineered non-human antibody that has an amino acid sequence modified to increase homology to the sequence of a human antibody. Generally, all or part of the CDRs of a humanized antibody is derived from a non-human antibody (donor antibody), and all or part of the non-CDRs (e.g., variable region FRs and/or constant regions) is derived from a human immunoglobulin (receptor antibody). The humanized antibody generally retains or partially retains the desired properties of the donor antibody, including, but not limited to, antigen specificity, affinity, reactivity, the ability to increase the activity of immune cells, the ability to enhance immune response, and the like.

The term “variable region” herein refers to a region of a heavy or light chain of an antibody involved in the binding of the antibody to an antigen. The “heavy chain variable region” is used interchangeably with “VH” and “HCVR”, and the “light chain variable region” is used interchangeably with “VL” and “LCVR”. Heavy and light chain variable domains (VH and VL, respectively) of natural antibodies generally have similar structures, each of which contains four conservative framework regions (FRs) and three hypervariable regions (HVRs). See. e.g., Kindt et al., Kuby Immunology. 6th ed., W. H. Freeman and Co., p. 91 (2007). A single VH or VL domain may be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region as it is precisely complementary to an epitope in spatial structures, wherein the heavy chain variable region CDR may be abbreviated as HCDR and the light chain variable region CDR may be abbreviated as LCDR. The terms “framework region” or “FR” are used interchangeably and refer to those amino acid residues of an antibody heavy chain variable region or light chain variable region other than CDRs. Generally, a typical antibody variable region consists of 4 FRs and 3 CDRs in the following order: FR1-CDR1-FR2-CDR2-FR3-CDR3-FR4.

For further description of the CDRs, see Kabat et al., J. Biol. Chem., 252: 6609-6616 (1977); Kabat et al., United States Department of Health and Human Services. Sequences of proteins of immunological interest (1991); Chothia et al., J. Mol. Biol. 196: 901-917 (1987); Al-Lazikani B. et al., J. Mol. Biol., 273: 927-948 (1997); MacCallum et al., J. Mol. Biol. 262: 732-745 (1996); Abhinandan and Martin. Mol. Immunol., 45: 3832-3839 (2008); Lefranc M. P. et al., Dev. Comp. Immunol., 27: 55-77 (2003); and Honegger and Pluckthun. J. Mol. Biol., 309: 657-670 (2001). The “CDR” herein may be labeled and defined in a manner well known in the art, including, but not limited to, Kabat numbering scheme. Chothia numbering scheme, or IMGT numbering scheme: the tool websites used include, but are not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi #results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “conservative amino acid” herein generally refers to amino acids that belong to the same class or have similar characteristics (e.g., charge, side chain size, hydrophobicity, hydrophilicity, backbone conformation, and rigidity). Illustratively, the amino acids in each of the following groups belong to conservative amino acid residues of each other, and substitutions of amino acid residues within the groups belong to conservative amino acid substitutions:

Illustratively, the following six groups are examples of amino acids that are considered to be conservative replacements of each other:
1) alanine (A), serine(S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The term “identity” can be obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needlema and Wunsch algorithm ((1970) J. Mol. Biol., 48:444-453; available at www.gcg.com) which has been integrated into the GAP program of the GCG software package, using the Blossum 62 matrix or PAM250 matrix and gap weight of 16, 14, 12, 10, 8, 6, or 4 and length weight of 1, 2, 3, 4, 5, or 6. For another example, the percent identity between two nucleotide acid sequences is determined with the GAP program of the GCG software package (available at www.gcg.com), using the NWSgapdna.CMP matrix and gap weight of 40, 50, 60, 70, or 80 and length weight of 1, 2, 3, 4, 5, or 6. A particularly preferred parameter set (and one that should be used unless otherwise stated) is a Blossum62 scoring matrix with a gap penalty of 12, a gap extension penalty of 4, and a frameshift gap penalty of 5.

The percent identity between two amino acid sequences or nucleotide sequences can also be determined with a PAM120 weighted remainder table, a gap length penalty of 12, and a gap penalty of 4, using the E. Meyers and W. Miller algorithm ((1989) CABIOS, 4:11-17) which has been incorporated into the ALIGN program (version 2.0).

Additionally or alternatively, the nucleic acid sequences and protein sequences described herein can be further used as “query sequences” to perform searches against public databases to, e.g., identify other family member sequences or related sequences. For example, such searches can be performed using the NBLAST and XBLAST programs (version 2.0) of Altschul et al., (1990) J. Mol. Biol., 215:403-10. BLAST nucleotide searches can be performed using the NBLAST program, with a score of 100 and a word length of 12, to obtain nucleotide sequences homologous to the nucleic acid (SEQ ID NO: 1) molecule of the present disclosure. BLAST protein searches can be performed using the XBLAST program, with a score of 50 and a word length of 3, to obtain amino acid sequences homologous to the protein molecule of the present disclosure. To obtain gapped alignment results for the purpose of comparison, gapped BLAST can be used as described in Altschul et al. (1997) Nucleic Acids Res. 25:3389-3402. When using the BLAST and gapped BLAST programs, the default parameters of the respective programs (e.g., XBLAST and NBLAST) can be used. See www.ncbi.nlm.nih.gov.

The term “pharmaceutical composition” herein refers to a formulation that exists in a form allowing the biological activity of the active ingredient contained therein to be effective, and does not contain additional ingredients having unacceptable toxicity to a subject to which the pharmaceutical composition is administered. The purpose of the pharmaceutical composition is to maintain the stability of the active ingredient of the antibody and promote the administration to an organism, which facilitates the absorption of the active ingredient, thereby exerting biological activity. As used herein, “pharmaceutical composition” and “formulation” are not mutually exclusive. In some embodiments, the term “pharmaceutical composition” herein includes antibodies and antibody-derived molecules such as immunoconjugates.

The term “immunoconjugate” herein refers to a polypeptide molecule containing at least one effector molecule and at least one antibody or a functional fragment thereof. The term “effector molecule” herein is a part of an immunoconjugate, which is intended to have a desired effect on cells targeted by the immunoconjugate. An effector molecule is also referred to as an effector moiety (EM), a therapeutic agent, a diagnostic agent, a tracer, or a similar term.

The term “stable” herein refers to a formulation in which the protein in the formulation substantially retains its physical and/or chemical stability and/or biological activity upon storage. The shelf life is generally selected based on a predetermined shelf life of the pharmaceutical composition. Various analytical techniques for measuring protein stability are available in the art.

A stable pharmaceutical antibody formulation is one in which no significant change is observed under the following conditions: stored at refrigeration temperature (2-8° C.) for at least 3 months, preferably 6 months, and more preferably 1 year. In addition, a stable liquid formulation includes a liquid formulation that exhibits desirable characteristics after storage at temperatures including 25° C., and 40° C. for periods of time including 1 month and 3 months. For example, a stable formulation: the pharmaceutical antibody formulation is colorless, or clear to slightly milky white upon visual analysis. The concentration, pH, and osmolality of the formulation have no more than +10% variation. Typically no more than about 10%, preferably no more than about 5% truncation is observed. Typically no more than about 10%, preferably no more than about 5% of the aggregate is formed.

The antibody “retains its physical stability” in the pharmaceutical formulation if it does not exhibit a significant increase in aggregation, precipitation and/or denaturation after visual inspection of color and/or clarity, or as measured by UV light scattering, size exclusion chromatography (SEC), and dynamic light scattering (DLS). The antibody “retains its chemical stability” in the pharmaceutical formulation if it does not exhibit significant chemical changes. Chemical stability can be assessed by detecting and quantifying the chemically altered form of the protein. Degradation processes that often alter the chemical structure of proteins include hydrolysis or truncation (assessed by methods such as size exclusion chromatography and SDS-PAGE), oxidation (assessed by methods such as peptide spectroscopy combined with mass spectrometry or MALDI/TOF/MS), deamidation (assessed by methods such as ion exchange chromatography, capillary isoelectric focusing, peptide spectroscopy, and isoaspartic acid measurement), and isomerization (assessed by measuring isoaspartic acid content, peptide spectroscopy, etc.).

The antibody “retains its biological activity” in the pharmaceutical formulation if its biological activity at a given time is within a predetermined range of the biological activity exhibited when the pharmaceutical formulation is prepared. The biological activity of the antibody can be determined, for example, by an antigen-binding assay.

The term “buffer” herein encompasses those agents that maintain the pH of the solution of the pharmaceutical composition of the present disclosure within an acceptable range, including acetate, succinate, gluconate, histidine, oxalate, lactate, phosphate, citrate, tartrate, fumarate, glycylglycine, and other organic acid buffers. The term “non-reducing sugar” herein may include at least one selected from trehalose or sucrose.

The term “non-ionic surfactant” is selected from non-ionic water-soluble monoglycerides, non-ionic water-soluble diglycerides, non-ionic water-soluble triglycerides, non-ionic water-soluble polyethylene glycol mono fatty acid esters, non-ionic water-soluble polyethylene glycol di fatty acid esters, non-ionic water-soluble sorbitol fatty acid esters, non-ionic glycosylated glycerides, non-ionic water-soluble triblock copolymers, and combinations thereof. In some embodiments, the non-ionic surfactant is polysorbate 80 (polyoxyethylene (20) sorbitol monooleate). The term “Tm value” herein refers to a temperature at which a protein is thermally denatured, i.e., a temperature at which half of the protein is unfolded and the spatial structure of the protein is destroyed, so that the higher the Tm value, the higher the thermal stability of the protein.

The term “treatment” herein refers to surgical or therapeutic treatment for the purpose of preventing or slowing (reducing) the progression of an undesired physiological or pathological change, e.g., a cancer, in a subject being treated. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission of state of disease (whether partial or total), whether detectable or undetectable. Subjects in need of treatment include those already with a disorder or disease, as well as those who are susceptible to a disorder or disease or those who intend to prevent a disorder or disease. When referring to terms such as slowing, alleviation, decrease, palliation, and remission, their meanings also include elimination, disappearance, nonoccurrence, etc.

The term “patient” herein refers to an organism that receives treatment for a particular disease or disorder described herein. Examples of subjects and patients include mammals, such as humans, primates (e.g., monkey), or non-primate mammals, that receive treatment for a disease or disorder.

The term “effective amount” herein refers to an amount of a therapeutic agent that is effective to prevent or alleviate symptoms of a disease or the progression of the disease when administered to a cell, tissue, or subject alone or in combination with another therapeutic agent. “Effective amount” also refers to an amount of a compound that is sufficient to alleviate symptoms, e.g., to treat, cure, prevent, or alleviate related medical disorders, or to increase the rates at which such disorders are treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially, or simultaneously.

The term “cancer” herein refers to or describes a physiological condition in mammals that is typically characterized by unregulated cell growth. Included in this definition are benign and malignant cancers. The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

### DETAILED DESCRIPTION

The present disclosure will be further described with reference to specific examples, and the advantages and features of the present disclosure will become more apparent with the description. Experimental procedures without specified conditions in the examples are conducted according to conventional conditions or conditions recommended by the manufacturers. Reagents or instruments without specified manufacturers used herein are conventional products that are commercially available. The examples are exemplary only and do not limit the scope of the present disclosure in any way. It will be understood by those skilled in the art that various changes or substitutions in form and details may be made to the technical solutions of the present disclosure without departing from the spirit and scope of the present disclosure, and that these changes and substitutions shall fall within the scope of the present disclosure.

### Noun Interpretation:

| Abbreviation | Meaning |
|---|---|
| T0 | Representing “starting point of investigation” |
| W | Representing “week”, e.g., “1 W” represents “1 week” |
| M | Representing “month”, e.g., “3 M” represents “3 |
| | months” |
| C | Representing cycle number of freezing-thawing, e.g., “3 C” |
| | represents 3 cycles of freezing-thawing, and “5 C” |
| | represents 5 cycles of freezing-thawing |
| D | Representing “day”, e.g., “7 D” represents “7 days” |
| H | Representing “hour” |
| ND | Representing “not detected” |
| F/T | Freeze/Thaw, Freeze-Thaw |
| SE-HPLC | Size-exclusion high-performance liquid chromatography |
| CEX | Cation exchange chromatography |
| CE-SDS | Non-reduced capillary electrophoresis-sodium dodecyl |
| (NR) | sulfate |
| CE-SDS (R) | Reduced capillary electrophoresis-sodium dodecyl sulfate |
| DLS | Dynamic light scattering |
| DSF | Differential scanning fluorimetry |
| ELISA | Enzyme-linked immunosorbent assay |

The ELISA binding assay described below was performed using the following method, unless otherwise indicated:

A PVRIG/TIGIT bispecific antibody is capable of specifically binding to hTIGIT-mFc at one end and to hPVRIG-His at the other end. A 96-well plate was pre-coated with an antigen (hTIGIT-mFc) (manufacturer: Acro, Cat. No. TIT-H5253), and the PVRIG/TIGIT bispecific antibody diluted in gradient to different concentrations was added to bind to the antigen on the ELISA plate. The plate was washed to remove various unbound components, and then a primary antibody (hPVRIG-His) (manufacturer: Acro, Cat. No. PVG-H52H4) was added for incubation. After the plate was washed again to remove the unbound primary antibody, a secondary antibody (His tag horseradish peroxidase-conjugated antibody; manufacturer: R&D, Cat. No. MAB050H) was added for incubation. After the incubation, the plate was washed to remove the unbound secondary antibody, and finally, TMB was added for color developing. The reaction was stopped using a stop solution, and the OD value was read using a microplate reader.

### Example 1. Construction, Expression, and Purification of PVRIG/TIGIT Bispecific Antibodies

### 1.1 Construction of PVRIG/TIGIT Bispecific Antibodies

Two humanized anti-PVRIG VHH antibodies (PVRIG-A50-H1b and PVRIG-A105-H1) and two humanized anti-TIGIT monoclonal antibodies (TIGIT-002-H4L3 and TIGIT-005-H2L1d) were used. The humanized anti-PVRIG VHH antibodies were connected to the N-terminus of the heavy chains of the humanized anti-TIGIT antibodies using a G4S linker peptide to produce humanized anti-PVRIG×TIGIT bispecific antibodies, named LC-BsAb-002, LC-BsAb-006, LC-BsAb-009, and LC-BsAb-010. Table 1 shows the sequences of the heavy chain fusion polypeptides (HC) and light chain polypeptides (LC) of the 4 bispecific antibodies. Table 2 shows the variable region sequences of the bispecific antibodies. Table 3 shows the CDR sequences of the bispecific antibodies according to Kabat.

TABLE 1
Sequences of fusion polypeptides of bispecific antibodies

| HC/LC | Sequence No. | sequence |
|---|---|---|
| LC of | SEQ ID NO: | DIVMTQSPDSLAVSLGERATINCKASQNVRTAVAWYQQKPGQSP |
| LC-BsAb-002 | 1 | KLMIYSASYRYTGVPDRFSGSGSGTDFTLTISSLQAEDVAVYYC |
| | | QQYYTTPWTFGGGTKVEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| | | Note: TIGIT-002-H4L3 VL (single underlined) |
| HC of | SEQ ID NO: | EVQLVESGGGLVQPGGSLRLSCAASGFTFSYYDMSWVRQAPG |
| LC-BsAb-002 | 2 | KGLEWVSTINSDGGRTSYVDSVKGRFTISRDNSKNTLYLQMNS |
| | | LRAEDTAVYYCVEGDPHNEGLESLSLRDEGSWGQGTMVTVSSG |
| | | GGGSGGGGSGGGGSGGGGSEVQLQESGPGLVKPSETLSLTCAVS |
| | | GYSITSDSWNWIRQPPGKKLEYIGYISYSGNTYYNPSLKSRVTIS |
| | | RDTSKNQFSLKLSSVTAADTAVYYCARLDFSNYGGAVDYWGQ |
| | | GTTVTVSSASTKGPSVFPLAPSSKSTSGGTAALGCLVKDYFPEP |
| | | VTVSWNSGALTSGVHTFPAVLQSSGLYSLSSVVTVPSSSLGTQT |
| | | YICNVNHKPSNTKVDKKVEPKSCDKTHTCPPCPAPELLGGPSVF |
| | | LFPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKS |
| | | RWQQGNVFSCSVMHEALHNHYTQKSLSLSPGK |
| | | Note: PVRIG-A50-H1b (double underlined) + (G4S)4 linker (italic) + |
| | | TIGIT-002-H4L3 VH (single underlined) |
| LC of | SEQ ID NO: | DIVMTQSPDSLAVSLGERATINCKASQHVSNAVAWYQHKPGQS |
| LC-BsAb-006 | 3 | PKLLIYSASYRYTGVPDRFSGSGSGTDFTLTISSLQAEDVAVYYC |
| | | QQHYNTPHTFGGGTKVEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| | | Note: TIGIT-005-H2L1d VL (single underline) |
| HC of | SEQ ID NO: | EVQLVESGGGLVQPGGSLRLSCAASGFTESYYDMSWVRQAPG |
| LC-BsAb-006 | 4 | KGLEWVSTINSDGGRTSYVDSVKGRFTISRDNSKNTLYLQMNS |
| | | LRAEDTAVYYCVEGDPHNFGLESLSLRDFGSWGQGTMVTVSSG |
| | | GGGSGGGGSGGGGSGGGGSEVQLVQSGAEVKKPGASVKVSCK |
| | | ASGYAFTNYLIEWVRQAPGQRLEWMGVINPGSGGTNYKEKFK |
| | | GRVTITADKSSSTAYMELSSLRSEDTAVYYCARGEYFFFDYWGQ |
| | | GTTVTVSSASTKGPSVFPLAPSSKSTSGGTAALGCLVKDYFPEP |
| | | VTVSWNSGALTSGVHTFPAVLQSSGLYSLSSVVTVPSSSLGTQT |
| | | YICNVNHKPSNTKVDKKVEPKSCDKTHTCPPCPAPELLGGPSVF |
| | | LFPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKS |
| | | RWQQGNVFSCSVMHEALHNHYTQKSLSLSPGK |
| | | Note: PVRIG-A50-H1b (double underlined) + (G4S)4 linker (italic) + |
| | | TIGIT-005-H2L1d VH (single underlined) |
| LC of | SEQ ID NO: | DIVMTQSPDSLAVSLGERATINCKASQNVRTAVAWYQQKPGQSP |
| LC-BsAb-009 | 5 | KLMIYSASYRYTGVPDRFSGSGSGTDFTLTISSLQAEDVAVYYC |
| | | QQYYTTPWTFGGGTKVEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| | | Note: TIGIT-002-H4L3 VL (single underlined) |
| HC of | SEQ ID NO: | EVQLVESGGGLVQPGGSLRLSCAASGRTFDRHTMSWFRQAPGK |
| LC-BsAb-009 | 6 | EREFVATASRIPGDTYYVDSVKGRFTISRDNAKNSLYLQMNSLR |
| | | AEDTAVYYCAATSAYCSEVDCYEKGSWYDNWGQGTMVTVSS |
| | | GGGGSGGGGSGGGGSGGGGSEVQLQESGPGLVKPSETLSLTCA |
| | | VSGYSITSDSWNWIRQPPGKKLEYIGYISYSGNTYYNPSLKSRV |
| | | TISRDTSKNQFSLKLSSVTAADTAVYYCARLDFSNYGGAVDYW |
| | | GQGTTVTVSSASTKGPSVFPLAPSSKSTSGGTAALGCLVKDYFP |
| | | EPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSV |
| | | FLFPPKPKDTLMISRTPEVTCVVVDVSHEDPEVKFNWYVDGVE |
| | | VHNAKTKPREEQYNSTYRVVSVLTVLHQDWLNGKEYKCKVSN |
| | | KALPAPIEKTISKAKGQPREPQVYTLPPSRDELTKNQVSLTCLVK |
| | | GFYPSDIAVEWESNGQPENNYKTTPPVLDSDGSFFLYSKLTVDK |
| | | SRWQQGNVFSCSVMHEALHNHYTQKSLSLSPGK |
| | | Note: PVRIG-A105-H1 (double underlined) + (G4S)4 linker (italic) + |
| | | TIGIT-002-H4L3 VH (single underlined) |
| LC of | SEQ ID NO: | DIVMTQSPDSLAVSLGERATINCKASQHVSNAVAWYQHKPGQS |
| LC-BsAb-010 | 7 | PKLLIYSASYRYTGVPDRFSGSGSGTDFTLTISSLQAEDVAVYYC |
| | | QQHYNTPHTFGGGTKVEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| | | Note: TIGIT-005-H2L1d VL (single underline) |
| HC of | SEQ ID NO: | EVQLVESGGGLVQPGGSLRLSCAASGRTFDRHTMSWFRQAPGK |
| LC-BsAb-010 | 8 | EREFVATASRIPGDTYYVDSVKGRFTISRDNAKNSLYLQMNSLR |
| | | AEDTAVYYCAATSAYCSEVDCYEKGSWYDNWGQGTMVTVSS |
| | | GGGGSGGGGSGGGGSGGGGSEVQLVQSGAEVKKPGASVKVSC |
| | | KASGYAFTNYLIEWVRQAPGQRLEWMGVINPGSGGTNYKEKF |
| | | KGRVTITADKSSSTAYMELSSLRSEDTAVYYCARGEYFFFDYWG |
| | | QGTTVTVSSASTKGPSVFPLAPSSKSTSGGTAALGCLVKDYFPE |
| | | PVTVSWNSGALTSGVHTFPAVLQSSGLYSLSSVVTVPSSSLGTQT |
| | | YICNVNHKPSNTKVDKKVEPKSCDKTHTCPPCPAPELLGGPSVF |
| | | LFPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKS |
| | | RWQQGNVFSCSVMHEALHNHYTQKSLSLSPGK |
| | | Note: PVRIG-A105-H1 (double underlined) + (G4S)4 linker (italic) + |
| | | TIGIT-005-H2L1d VH (single underlined) |

TABLE 2
Variable region sequences of bispecific antibodies

| Variable region | Sequence No. | sequence |
|---|---|---|
| PVRIG-A50-H1b | SEQ ID NO: 9 | EVQLVESGGGLVQPGGSLRLSCAASGFTFSYYDMSWV |
| | | RQAPGKGLEWVSTINSDGGRTSYVDSVKGRFTISRDN |
| | | SKNTLYLQMNSLRAEDTAVYYCVEGDPHNFGLESLSL |
| | | RDFGSWGQGTMVTVSS |
| PVRIG-A105-H1 | SEQ ID NO: 10 | EVQLVESGGGLVQPGGSLRLSCAASGRTFDRHTMSWF |
| | | RQAPGKEREFVATASRIPGDTYYVDSVKGRFTISRDNA |
| | | KNSLYLQMNSLRAEDTAVYYCAATSAYCSEVDCYEKG |
| | | SWYDNWGQGTMVTVSS |
| TIGIT-002-H4L3 VL | SEQ ID NO: 11 | DIVMTQSPDSLAVSLGERATINCKASQNVRTAVAWYQ |
| | | QKPGQSPKLMIYSASYRYTGVPDRFSGSGSGTDFTLTIS |
| | | SLQAEDVAVYYCQQYYTTPWTFGGGTKVEIK |
| TIGIT-002-H4L3 VH | SEQ ID NO: 12 | EVQLQESGPGLVKPSETLSLTCAVSGYSITSDSWNWIR |
| | | QPPGKKLEYIGYISYSGNTYYNPSLKSRVTISRDTSKNQ |
| | | FSLKLSSVTAADTAVYYCARLDFSNYGGAVDYWGQG |
| | | TTVTVSS |
| TIGIT-005-H2L1d | SEQ ID NO: 13 | DIVMTQSPDSLAVSLGERATINCKASQHVSNAVAWYQ |
| VL | | HKPGQSPKLLIYSASYRYTGVPDRFSGSGSGTDFTLTIS |
| | | SLQAEDVAVYYCQQHYNTPHTFGGGTKVEIK |
| TIGIT-005-H2L1d | SEQ ID NO: 14 | EVQLVQSGAEVKKPGASVKVSCKASGYAFTNYLIEWV |
| VH | | RQAPGQRLEWMGVINPGSGGTNYKEKFKGRVTITAD |
| | | KSSSTAYMELSSLRSEDTAVYYCARGEYFFFDYWGQG |
| | | TTVTVSS |

TABLE 3
KABAT analysis for bispecific antibodies

| Variable region | CDR1 | CDR2 | CDR3 |
|---|---|---|---|
| PVRIG-A50-H1b | YYDMS | TINSNGGRTSYVDSVKG | GDPHNFGLENLSLRDFGS |
| | SEQ ID NO: 15 | SEQ ID NO: 16 | SEQ ID NO: 17 |
| PVRIG-A105-H1 | RHTMS | TASRIPGDTYYVDSVKG | TSAYCSEVDCYEKGSWYDN |
| | SEQ ID NO: 18 | SEQ ID NO: 19 | SEQ ID NO: 20 |
| Variable region | HCDR1 | HCDR2 | HCDR3 |
| TIGIT-002-H4L3 | SDSWN | YISYSGNTYYNPSLKS | LDFSNYGGAVDY |
| VH | SEQ ID NO: 21 | SEQ ID NO: 22 | SEQ ID NO: 23 |
| TIGIT-005-H2L1d | NYLIE | VINPGSGGTNYKEKFKG | GEYFFFDY |
| VH | SEQ ID NO: 24 | SEQ ID NO: 25 | SEQ ID NO: 26 |
| Variable region | LCDR1 | LCDR2 | LCDR3 |
| TIGIT-002-H4L3 | KASQNVRTAVA | SASYRYT | QQYYTTPWT |
| VL | SEQ ID NO: 27 | SEQ ID NO: 28 | SEQ ID NO: 29 |
| TIGIT-005-H2L1d | KASQHVSNAVA | SASYRYT | QQHYNTPHT |
| VL | SEQ ID NO: 30 | SEQ ID NO: 31 | SEQ ID NO: 32 |

### 1.2 Expression of PVRIG/TIGIT Bispecific Antibodies

The PVRIG/TIGIT bispecific antibody genes were transfected into CHO-K1 cells by adopting a gene engineering technology, and clones with higher yield were screened out under MSX pressure and subcultured in a CD CHO culture medium. The PVRIG/TIGIT bispecific antibodies were expressed using a fed-batch culture mode. The basic culture medium was CHO CDP9 from OPM, and the culture period was 14-16 days, with the reactor control parameters of pH 6.6-7.2, dissolved oxygen (DO) of ≥20%, rotation speed of 75 rpm-80 rpm, and initial culture temperature of 36.0° C.-37.0° C.; on day 6 of culturing, the culture temperature was lowered to 33.5° C.-34.5° C. until harvest.

The feeding media were XF04 and CD FS08 from OPM and were added every other day starting from day 3, with feeding volumes of 5% and 0.5% of the volume, respectively, until harvest.

### 1.3 Purification of PVRIG/TIGIT Bispecific Antibodies

The purification of the PVRIG/TIGIT bispecific antibodies was performed sequentially through multi-step chromatography, concentration, and filtration unit operations. The supernatant harvest was captured by Protein A affinity chromatography (AT Protein A Diamond Plus). The captured antibody solution was treated with a low pH incubation to inactivate potential viruses. After the antibody solution was neutralized, the precipitate was removed by depth filtration. Then, anion exchange chromatography (Diamond Q) was performed to remove impurities such as HCD, HCP, and shed Protein A, and then cation exchange chromatography (Diamond S) was performed to remove impurities such as HCP and aggregates. The fusion protein solution was filtered through a nanofiltration membrane to remove potential endogenous and exogenous viruses. Then, the antibody solution was concentrated by an ultrafiltration membrane, and the buffer was exchanged, thereby completing the purification to obtain a PVRIG/TIGIT bispecific antibody protein stock solution.

### 1.4 Assay on Affinity of PVRIG/TIGIT Bispecific Antibodies for Human, Cynomolgus Monkey, and Mouse TIGIT and PVRIG Proteins by BIAcore

A Protein A chip was adopted in this experiment, and the time required for the chip to capture the diluted antibodies was measured by manual run so that the saturated antigen binding Rmax was 50 RU. The human, cynomolgus monkey, and mouse TIGIT and PVRIG proteins were diluted in gradient to 20, 10, 5, 2.5, and 1.25 nM. The affinity of the antibodies for the antigens was determined using multi-cycle kinetics. In each cycle, after the antibodies were injected, gradient concentrations of the human, cynomolgus monkey, and mouse TIGIT and PVRIG proteins were injected to ensure that the antigens and the antibodies were bound and dissociated. After each cycle, the Protein A chip was regenerated using Glycine pH 1.5 (to remove proteins on the chip). BIAcore T200 analysis software was used to fit the affinity KD of the antibodies for the antigens. It can be seen from the results in Table 4 that the 2 bispecific antibodies specifically bind to the human and cynomolgus monkey TIGIT and PVRIG proteins with high affinity levels, but do not bind to the mouse TIGIT and PVRIG proteins.

TABLE 4
Affinity of PVRIG/TIGIT bispecific antibodies for
TIGIT and PVRIG proteins from different species

| | | Binding kinetics | | | | |
|---|---|---|---|---|---|---|
| | | | | | | Capture |
| | | ka | kd | KD | Rmax | Level |
| Antibody | Antigen | (1/Ms) | (1/s) | (M) | (RU) | (RU) |
| LC-BsAb-002 | hTIGIT | 1.75E+06 | 2.59E−04 | 1.48E−10 | 100.3 | 727 |
| | cynoTIGIT | 4.92E+05 | 6.43E−03 | 1.31E−08 | 112.9 | 726 |
| | mTIGIT | / | / | / | 0.1 | 726 |
| | hPVRIG | 3.09E+05 | 5.87E−05 | 1.90E−10 | 106.6 | 557 |
| | cynoPVRIG | 3.57E+04 | 1.31E−04 | 3.66E−09 | 109.7 | 659 |
| | mPVRIG | / | / | / | 0.1 | 579 |
| LC-BsAb-006 | hTIGIT | 1.65E+06 | 2.14E−04 | 1.30E−10 | 99.4 | 696 |
| | cynoTIGIT | 1.09E+06 | 1.84E−03 | 1.69E−09 | 108.0 | 695 |
| | mTIGIT | / | / | / | 0.9 | 695 |
| | hPVRIG | 3.40E+05 | 5.01E−05 | 1.48E−10 | 103.6 | 538 |
| | cynoPVRIG | 3.60E+04 | 1.42E−04 | 3.95E−09 | 109.8 | 631 |
| | mPVRIG | / | / | / | 0.2 | 1140 |

### 1.5 Assay on Co-Binding of PVRIG/TIGIT Bispecific Antibodies to Human TIGIT and PVRIG Proteins by BIAcore

BIAcore was used to characterize the simultaneous binding properties of the bispecific antibodies to the two antigens. Firstly, the antibodies LC-BsAb-002 and LC-BsAb-006 were captured by a Protein A chip, followed by separate injections of TIGIT and PVRIG his-tagged proteins, as well as separate sequential injections of TIGIT and PVIRIG and PVRIG and TIGIT. The binding signals of antibodies to antigens were recorded. Finally, the chip was regenerated using Glycine pH 1.5. The mobile phase was HBS-EP+ (10 mM HEPES. 150 mM NaCl. 3 mM EDTA, and 0.05% surfactant P20), and the flow rate was 30 μL/min. The binding time with different antigens was 300 s, the regeneration time was 30 s, and the detection temperature was 25° C. The analytical concentration of hTIGIT was 20 nM, and that of hPVRIG was 50 nM. The data were analyzed using BIAcore 8K analysis software (version number 2.0), and the capture levels of the antibodies and the binding responses (RU) of different antigens were recorded. The stoichiometric ratio of the antigen molecules to the antibody molecules was calculated according to the molecular weight of the antigen and the antibody, and the preliminary estimate was made of how many antigens an antibody molecule can bind to. To confirm the interaction between the antibody LC-BsAb-002 and the antigens TIGIT and PVRIG a four-step assay was conducted: binding to the antigen hTIGIT alone, binding to the antigen hPVRIG alone, binding to hTIGIT followed by hPVRIG and binding to hPVRIG followed by hTIGIT, with each antigen reaching a saturated state. The antigen-antibody binding curves were collected, and the capture levels of the two bispecific antibodies and the binding signals of TIGIT and PVIRIG in each experiment were recorded. The stoichiometric ratio of the antigen molecules to the antibody molecules was calculated accordingly. As shown in Table 5, the binding signals produced by the sequential injection of TIGIT and PVIRG were almost identical to those produced by the individual injections of TIGIT and PVIRG; moreover, the signals produced by the forward and reverse sequential injections of TIGIT and PVIRG were also almost identical. This indicates that LC-BsAb-002 and LC-BsAb-006 can bind simultaneously to hTIGIT and hPVRIG with no mutual influence existing between the two antigens. Considering the molecular weights of the antibodies and the antigens, as well as the capture levels of the antibodies and the binding levels of the antigens, it was preliminarily estimated that the stoichiometric ratio of TIGIT to LC-BsAb-002 was 1.76; the stoichiometric ratio of TIGIT to LC-BsAb-006 was 1.86; the stoichiometric ratio of PVIRIG to LC-BsAb-002 was 2.14, and the stoichiometric ratio of PVIRIG to LC-BsAb-006 was 2.18. The stoichiometric ratios of the two antigens to the antibodies were both close to 2. Considering the error introduced by the detection method, it is speculated that one LC-BsAb-002 or one LC-BsAb-006 bispecific antibody molecule can simultaneously bind to two TIGIT molecules and two PVRIG molecules.

TABLE 5
BIAcore binding of PVRIG/TIGIT bispecific antibodies to TIGIT and PVRIG proteins

| | | Antigen 01 | | | Antigen 02 | | |
|---|---|---|---|---|---|---|---|
| | Capture | | Binding | Antibody- | | Binding | Antibody- |
| | Level | | Responses | Antigen | | Responses | Antigen |
| Antibody | (RU) | Name | (RU) | Stoichiometry | Name | (RU) | Stoichiometry |
| LC-BsAb-002 | 673.6 | TIGT | 94.96 | 1.76 | / | / | / |
| | 673.1 | PVRIG | 128.67 | 2.14 | / | / | / |
| | 673.3 | TIGT | 94.87 | 1.76 | PVRIG | 121.11 | 2.02 |
| | 673.7 | PVRIG | 128.79 | 2.15 | TIGT | 91.28 | 1.69 |
| LC-BsAb-006 | 603.7 | TIGT | 89.83 | 1.86 | / | / | / |
| | 602.9 | PVRIG | 117.08 | 2.18 | / | / | / |
| | 603.4 | TIGT | 89.83 | 1.86 | PVRIG | 109.42 | 2.04 |
| | 602.3 | PVRIG | 116.96 | 2.18 | TIGT | 83.88 | 1.74 |

### 1.6 Killing Effect of PVRIG/TIGIT Bispecific Antibodies

The PVRIG/TIGIT bispecific antibodies LC-BsAb-002 and LC-BsAb-006 can effectively promote the killing of NK cells to target cells WIDR and can also show concentration-dependent ADCC killing to Treg cells.

The A375 cells were inoculated into the mice, and when the tumors had grown to an appropriate size, the PVRIG/TIGIT bispecific antibodies LC-BsAb-002 and LC-BsAb-006 were administered. The results showed that the bispecific antibodies had a significant inhibitory effect on the growth of A375 tumors.

### Example 2. pH/Buffer System Screening

### 2.1 Study Protocol

(1) 12 different buffer formulas F1-F12 were designed, and the specific information of the candidate formulas is shown in Table 1. (2) The PVRIG/TIGIT bispecific antibody LC-BsAb-002 protein stock solution obtained in Example 1 was exchanged into F1-F12 using an ultrafiltration concentration centrifuge tube of Millipore, and the protein concentration was adjusted to 50 mg/mL. (3) Through accelerated stability tests at 25° C., and 40° C., the stability of the PVRIG/TIGIT bispecific antibody in the 12 candidate formulas was comprehensively investigated to select an optimal pH/buffer system for subsequent development of excipients. The assessment indicators included appearance, pH, protein concentration, dynamic light scattering (DLS), thermal stability (DSF), whole-column imaging capillary isoelectric focusing electrophoresis (iCIEF), purity liquid (size-exclusion high-performance chromatography (SE-HPLC), non-reducing capillary electrophoresis-sodium dodecyl sulfate (CE-SDS (NR&R))), and binding activity (ELISA-Binding). The investigation protocol is detailed in Table 6.

TABLE 6
Formulas and investigation protocol for pH/buffer system screening

| Formula | Buffer | | | 25° C. | | 40° C. | | |
|---|---|---|---|---|---|---|---|---|
| No. | system, pH | | T0 | 2 W | 4 W | 1 W | 2 W | 4 W |
| F1 | 20 mM acetic | 4.5 | X, Y, | X | X, Y | X | X | X, Y |
| F2 | acid-sodium | 5.0 | Z | | | | | |
| F3 | acetate | 5.5 | | | | | | |
| F4 | 20 mM citric | 5.0 | | | | | | |
| F5 | acid-sodium | 5.5 | | | | | | |
| F6 | citrate | 6.0 | | | | | | |
| F7 | 20 mM | 5.5 | | | | | | |
| F8 | histidine- | 6.0 | | | | | | |
| F9 | histidine | 6.5 | | | | | | |
| | hydrochloride | | | | | | | |
| F10 | 20 mM | 6.5 | | | | | | |
| F11 | disodium | 7.0 | | | | | | |
| F12 | hydrogen | 7.5 | | | | | | |
| | phosphate- | | | | | | | |
| | sodium | | | | | | | |
| | dihydrogen | | | | | | | |
| | phosphate | | | | | | | |

Note:
X = appearance, pH, protein concentration, DLS, iCIEF, SE-HPLC, CE-SDS (NR&R);
Y = ELISA-Binding;
Z = DSF.
T0 = investigation start point; W = week; the same below.

### 2.2 Screening Results

The major results of the pH/buffer system screening are summarized in Tables 7-16 below:

TABLE 7
Appearance detection results for pH/buffer system screening

| Formula | | 25° C. | | 40° C. | | |
|---|---|---|---|---|---|---|
| No. | T0 | 2 W | 4 W | 1 W | 2 W | 4 W |
| F1 | Colorless, | Colorless, | Colorless, | Colorless, | Colorless, | Colorless, |
| | clear | clear | clear | clear | clear | clear |
| | No visible | No visible | No visible | No visible | No visible | Fibrous |
| | particles | particles | particles | particles | particles | particles |
| F2 | Colorless, | Colorless, | Colorless, | Colorless, | Colorless, | Colorless, |
| | slightly | slightly | clear | slightly | slightly | clear |
| | opalescent | opalescent | No visible | opalescent | opalescent, | Fibrous |
| | No visible | No visible | particles | No visible | no visible | particles |
| | particles | particles | | particles | particles | |
| F3 | Colorless, | Colorless, | Colorless, | Colorless, | Colorless, | Colorless, |
| | slightly | slightly | slightly | slightly | slightly | slightly |
| | opalescent | opalescent | opalescent, | opalescent, | opalescent | opalescent |
| | No visible | No visible | fibrous | small amount | Fibrous | Fibrous |
| | particles | particles | particles | of fibrous | particles | particles |
| | | | | particles | | |
| F4 | Colorless, | Colorless, | Colorless, | Colorless, | Colorless, | Colorless, |
| | strongly | strongly | strongly | strongly | strongly | strongly |
| | opalescent, | opalescent, | opalescent, | opalescent, | opalescent, | opalescent, |
| | fibrous | fibrous | fibrous | fibrous | fibrous | fibrous |
| | particles | particles | particles | particles | particles | particles |
| F5 | Colorless, | Colorless, | Colorless, | Colorless, | Colorless, | Colorless, |
| | strongly | strongly | strongly | strongly | strongly | strongly |
| | opalescent, | opalescent, | opalescent, | opalescent, | opalescent, | opalescent, |
| | fibrous | fibrous | fibrous | fibrous | fibrous | fibrous |
| | particles | particles | particles | particles | particles | particles |
| F6 | Colorless, | Colorless, | Colorless, | Colorless, | Colorless, | Colorless, |
| | strongly | strongly | strongly | strongly | strongly | strongly |
| | opalescent, | opalescent, | opalescent, | opalescent, | opalescent, | opalescent, |
| | fibrous | fibrous | fibrous | fibrous | fibrous | fibrous |
| | particles | particles | particles | particles | particles | particles |
| F7 | Colorless, | Colorless, | Colorless, | Colorless, | Colorless, | Colorless, |
| | slightly | slightly | slightly | slightly | slightly | slightly |
| | opalescent | opalescent | opalescent, | opalescent | opalescent | opalescent, |
| | No visible | No visible | small amount | No visible | No visible | fibrous |
| | particles | particles | of fibrous | particles | particles | particles |
| | | | particles | | | |
| F8 | Colorless, | Colorless, | Colorless, | Colorless, | Colorless, | Colorless, |
| | slightly | slightly | slightly | slightly | slightly | slightly |
| | opalescent | opalescent | opalescent, | opalescent | opalescent | opalescent, |
| | No visible | No visible | small amount | No visible | No visible | fibrous |
| | particles | particles | of fibrous | particles | particles | particles |
| | | | particles | | | |
| F9 | Colorless, | Colorless, | Colorless, | Colorless, | Colorless, | Colorless, |
| | slightly | slightly | slightly | slightly | slightly | slightly |
| | opalescent | opalescent | opalescent | opalescent | opalescent | opalescent, |
| | No visible | No visible | Fibrous | No visible | Fibrous | fibrous |
| | particles | particles | particles | particles | particles | particles |
| F10 | Colorless, | Colorless, | Colorless, | Colorless, | Colorless, | Colorless, |
| | strongly | strongly | slightly | strongly | strongly | slightly |
| | opalescent, | opalescent, | opalescent, | opalescent, no | opalescent, | opalescent, |
| | no visible | fibrous | fibrous | visible | fibrous | fibrous |
| | particles | particles | particles | particles | particles | particles |
| F11 | Colorless, | Colorless, | Colorless, | Colorless, | Colorless, | Colorless, |
| | strongly | strongly | slightly | strongly | strongly | slightly |
| | opalescent, | opalescent, | opalescent, | opalescent, no | opalescent, | opalescent, |
| | no visible | fibrous | fibrous | visible | fibrous | fibrous |
| | particles | particles | particles | particles | particles | particles |
| F12 | Colorless, | Colorless, | Colorless, | Colorless, | Colorless, | Colorless, |
| | strongly | strongly | slightly | strongly | strongly | slightly |
| | opalescent, | opalescent, | opalescent, | opalescent, no | opalescent, | opalescent, |
| | no visible | fibrous | fibrous | visible | fibrous | fibrous |
| | particles | particles | particles | particles | particles | particles |

TABLE 8
pH detection results for pH/buffer system screening

| | | |
|---|---|---|
| Formula | 25° C. | 40° C. |

| No. | T0 | 2 W | 4 W | 1 W | 2 W | 4 W |
|---|---|---|---|---|---|---|
| F1 | 4.8 | 4.8 | 4.8 | 4.8 | 4.8 | 4.8 |
| F2 | 5.2 | 5.2 | 5.2 | 5.2 | 5.2 | 5.2 |
| F3 | 5.6 | 5.6 | 5.6 | 5.6 | 5.6 | 5.6 |
| F4 | 5.1 | 5.1 | 5.1 | 5.1 | 5.1 | 5.1 |
| F5 | 5.5 | 5.5 | 5.5 | 5.5 | 5.5 | 5.5 |
| F6 | 5.9 | 5.9 | 6.0 | 5.9 | 5.9 | 5.9 |
| F7 | 5.6 | 5.6 | 5.6 | 5.6 | 5.5 | 5.6 |
| F8 | 6.1 | 6.1 | 6.1 | 6.1 | 6.0 | 6.2 |
| F9 | 6.5 | 6.5 | 6.5 | 6.5 | 6.5 | 6.5 |
| F10 | 6.4 | 6.4 | 6.5 | 6.5 | 6.4 | 6.4 |
| F11 | 6.9 | 6.9 | 6.9 | 6.9 | 6.9 | 6.9 |
| F12 | 7.3 | 7.3 | 7.3 | 7.3 | 7.3 | 7.3 |

TABLE 9
Protein concentration (mg/mL) detection
results for pH/buffer system screening

| | | |
|---|---|---|
| Formula | 25° C. | 40° C. |

| No. | T0 | 2 W | 4 W | 1 W | 2 W | 4 W |
|---|---|---|---|---|---|---|
| F1 | 50.1 | 50.2 | 50.3 | 50.4 | 50.1 | 50.3 |
| F2 | 50.3 | 50.4 | 50.5 | 50.5 | 50.4 | 50.5 |
| F3 | 51.0 | 50.9 | 50.9 | 51.2 | 51.2 | 51.1 |
| F4 | 50.6 | 51.0 | 50.6 | 50.7 | 50.6 | 50.7 |
| F5 | 50.1 | 50.3 | 50.1 | 50.2 | 49.3 | 50.2 |
| F6 | 50.7 | 50.8 | 50.8 | 50.9 | 50.3 | 50.9 |
| F7 | 49.6 | 49.7 | 49.7 | 50.0 | 49.8 | 49.9 |
| F8 | 50.0 | 50.1 | 50.2 | 50.2 | 50.4 | 50.3 |
| F9 | 50.2 | 50.3 | 50.4 | 50.3 | 50.3 | 50.3 |
| F10 | 49.9 | 50.2 | 50.2 | 50.6 | 50.0 | 50.2 |
| F11 | 49.8 | 50.0 | 49.9 | 50.4 | 50.1 | 50.0 |
| F12 | 50.2 | 50.4 | 50.3 | 50.5 | 50.2 | 50.4 |

TABLE 10
DLS (diameter, nm) detection results
for pH/buffer system screening

| | | |
|---|---|---|
| Formula | 25° C. | 40° C. |

| No. | T0 | 2 W | 4 W | 1 W | 2 W | 4 W |
|---|---|---|---|---|---|---|
| F1 | 6.2 | 5.7 | 5.7 | 6.2 | 5.9 | 7.4 |
| F2 | 9.4 | 9.0 | 9.2 | 9.8 | 9.1 | 10.1 |
| F3 | 12.1 | 12.3 | 12.3 | 13.5 | 12.5 | 13.9 |
| F4 | 22.6 | 23.8 | 24.4 | 24.8 | 23.1 | 27.8 |
| F5 | 23.3 | 23.3 | 27.6 | 29.7 | 25.4 | 27.6 |
| F6 | 23.2 | 23.6 | 27.9 | 30.2 | 22.7 | 27.2 |
| F7 | 12.6 | 11.9 | 13.4 | 14.3 | 12.3 | 13.3 |
| F8 | 13.8 | 13.8 | 14.1 | 15.5 | 13.7 | 16.5 |
| F9 | 15.7 | 15.9 | 16.1 | 16.1 | 15.8 | 18.9 |
| F10 | 22.7 | 22.9 | 23.5 | 23.5 | 22.1 | 27.7 |
| F11 | 20.8 | 23.5 | 24.7 | 24.0 | 22.7 | 27.7 |
| F12 | 19.8 | 22.1 | 22.1 | 22.5 | 20.9 | 23.8 |

TABLE 11
DSF (° C.) detection results at T0
sampling point for pH/buffer system screening

T0

| | Melting temperature | Melting temperature |
|---|---|---|
| Formula No. | 1 (Tm1) | 2 (Tm2) |
| F1 | 68.6 | 78.3 |
| F2 | 68.8 | 77.9 |
| F3 | 68.9 | 77.9 |
| F4 | 68.1 | 78.4 |
| F5 | 68.3 | 78.3 |
| F6 | 68.6 | 78.3 |
| F7 | 68.9 | 78.9 |
| F8 | 69.0 | 78.1 |
| F9 | 68.9 | 77.9 |
| F10 | 69.1 | 78.2 |
| F11 | 69.0 | 78.1 |
| F12 | 69.1 | 78.0 |

TABLE 12
iCIEF (%) detection results for pH/butter system screening

| | | | | 25° C. | | | | | | 40° C. | | | | | | | | |
|---|---|---|---|---|---|---|---|---|---|---|---|---|---|---|---|---|---|---|
| Formula | T0 | | | 2 W | | | 4 W | | | 1 W | | | 2 W | | | 4 W | | |
| No. | MP | AP | BP | MP | AP | BP | MP | AP | BP | MP | AP | BP | MP | AP | BP | MP | AP | BP |
| F1 | 53.2 | 43.1 | 3.8 | 54.4 | 37.6 | 7.9 | 49.4 | 39.7 | 10.9 | 39.9 | 44.5 | 15.6 | 30.7 | 50.7 | 18.5 | 18.0 | 53.8 | 28.2 |
| F2 | 53.2 | 42.6 | 4.3 | 55 | 38.2 | 6.8 | 49.9 | 41.1 | 9.0 | 42.8 | 43.5 | 13.8 | 33.3 | 50.7 | 15.9 | 23.6 | 54.1 | 22.3 |
| F3 | 53 | 43.1 | 3.9 | 56.3 | 37.8 | 6 | 51.3 | 40.7 | 7.9 | 46.4 | 43.2 | 10.5 | 36 | 49.1 | 14.8 | 25.8 | 53.6 | 20.5 |
| F4 | 52.5 | 43.5 | 4.1 | 54.4 | 38.7 | 6.8 | 47.1 | 42.6 | 10.1 | 38.6 | 48.3 | 13.1 | 33.5 | 49.6 | 16.7 | 19.9 | 57.4 | 22.7 |
| F5 | 52.5 | 43.4 | 4.1 | 52.9 | 40.9 | 6.3 | 50.5 | 41.3 | 8.3 | 41.5 | 46.6 | 11.8 | 37.8 | 46.9 | 15.2 | 26.0 | 55.3 | 18.8 |
| F6 | 51.6 | 43.5 | 4.9 | 54.7 | 40.4 | 5 | 51.5 | 41.5 | 7.2 | 45.5 | 45 | 9.5 | 38.4 | 49.3 | 12.4 | 26.4 | 56.1 | 17.6 |
| F7 | 53.3 | 42.5 | 4.3 | 51.5 | 42.8 | 5.7 | 51.3 | 40.9 | 7.8 | 44.9 | 42.7 | 12.4 | 35.1 | 49.4 | 15.4 | 27.2 | 52.3 | 20.3 |
| F8 | 53.8 | 42.5 | 3.7 | 48.5 | 47.2 | 4.4 | 52.1 | 41.4 | 6.6 | 46.1 | 43.5 | 10.5 | 38.7 | 48.4 | 13 | 36.5 | 45.1 | 18.5 |
| F9 | 53.5 | 42.3 | 4.2 | 47.4 | 47.8 | 4.6 | 52.2 | 42.3 | 5.5 | 47.1 | 44.5 | 8.4 | 38.6 | 52 | 9.6 | 34.2 | 51.8 | 14.0 |
| F10 | 54.2 | 42.7 | 3 | 55.4 | 40 | 4.7 | 51.2 | 42.7 | 6.1 | 45.9 | 46 | 8.1 | 37.5 | 52.2 | 10.3 | 37.3 | 49.4 | 13.4 |
| F11 | 55.3 | 41 | 3.6 | 50 | 45.5 | 4.6 | 50.4 | 44.0 | 5.6 | 42.6 | 49.5 | 7.8 | 35.6 | 56.1 | 8.1 | 29.7 | 58.6 | 11.7 |
| F12 | 51.9 | 44.4 | 3.6 | 52.9 | 42.4 | 4.8 | 46.9 | 46.7 | 6.4 | 37.3 | 54.5 | 8.2 | 35.7 | 56.5 | 7.8 | 15.5 | 71.3 | 13.2 |

Note:
MP: main peak;
AP: acidic peak;
BP: basic peak

TABLE 13
SE-HPLC (%) detection results for pH/buffer system screening

| | | | | 25° C. | | | | | |
|---|---|---|---|---|---|---|---|---|---|
| Formula | T0 | | | 2 W | | | 4 W | | |
| No. | MP | HMW | LMW | MP | HMW | LMW | MP | HMW | LMW |
| F1 | 98 | 2 | ND | 97.9 | 1.9 | 0.2 | 97.8 | 1.7 | 0.5 |
| F2 | 97.9 | 2.1 | ND | 97.6 | 2.2 | 0.2 | 97.5 | 2.3 | 0.2 |
| F3 | 97.7 | 2.3 | ND | 97.4 | 2.6 | ND | 96.9 | 2.8 | 0.4 |
| F4 | 97.5 | 2.5 | ND | 97 | 2.8 | 0.2 | 96.6 | 2.8 | 0.6 |
| F5 | 97.4 | 2.6 | ND | 96.8 | 3 | 0.1 | 96.4 | 3.3 | 0.4 |
| F6 | 97.4 | 2.6 | ND | 96.4 | 3.5 | 0.1 | 95.8 | 3.9 | 0.3 |
| F7 | 97.9 | 2.1 | ND | 97.5 | 2.4 | 0.2 | 97.6 | 2.4 | 0 |
| F8 | 97.7 | 2.3 | ND | 97.2 | 2.8 | ND | 96.6 | 3.1 | 0.3 |
| F9 | 97.5 | 2.5 | ND | 96.6 | 3.3 | 0.1 | 95.9 | 3.7 | 0.4 |
| F10 | 96.9 | 3.1 | | 95.7 | 4.2 | 0.1 | 94.8 | 4.9 | 0.3 |
| F11 | 96.7 | 3.3 | ND | 95.2 | 4.7 | 0.1 | 94.3 | 5.3 | 0.5 |
| F12 | 96.6 | 3.4 | ND | 95.3 | 4.6 | 0.1 | 94.1 | 5.5 | 0.4 |
| | 40° C. | | | | | | | | |
| Formula | 1 W | | | 2 W | | | 4 W | | |
| No. | MP | HMW | LMW | MP | HMW | LMW | MP | HMW | LMW |
| F1 | 97.4 | 2.2 | 0.4 | 94.6 | 2.8 | 2.6 | 93.0 | 3.64 | 3.3 |
| F2 | 96.7 | 2.9 | 0.3 | 93.8 | 3.9 | 2.3 | 92.3 | 5.06 | 2.6 |
| F3 | 96 | 3.7 | 0.3 | 92.8 | 5 | 2.2 | 92.2 | 5.57 | 2.3 |
| F4 | 95.4 | 4.2 | 0.4 | 91.5 | 6 | 2.5 | 89.4 | 7.2 | 3.4 |
| F5 | 95.3 | 4.4 | 0.3 | 91.5 | 6.5 | 2 | 90.3 | 7.3 | 2.4 |
| F6 | 95 | 4.7 | 0.2 | 91.6 | 6.7 | 1.8 | 90.9 | 6.91 | 2.3 |
| F7 | 96.6 | 3.1 | 0.3 | 93.8 | 4.1 | 2.1 | 92.4 | 5.2 | 2.4 |
| F8 | 96 | 3.8 | 0.2 | 93.2 | 4.9 | 1.9 | 92.6 | 5.35 | 2.1 |
| F9 | 95.3 | 4.5 | 0.2 | 92.6 | 5.4 | 1.9 | 92.0 | 5.78 | 2.2 |
| F10 | 93.9 | 5.8 | 0.2 | 92.7 | 6.8 | 0.5 | 90.2 | 7.5 | 2.3 |
| F11 | 94.1 | 5.6 | 0.2 | 92.8 | 6.5 | 0.6 | 90.3 | 6.9 | 2.8 |
| F12 | 94.3 | 5.4 | 0.3 | 93.1 | 6 | 0.9 | 89.6 | 6.2 | 4.2 |

Note:
MP: main peak;
HMW: high molecular weight, high polymer;
LMW: low molecular weight, oligomer;
ND: not detected

TABLE 14
CE-SDS-NR (%) detection results for pH/buffer system screening

| | | | | 25° C. | | | | | |
|---|---|---|---|---|---|---|---|---|---|
| Formula | T0 | | | 2 W | | | 4 W | | |
| No. | MP | HMW | LMW | MP | HMW | LMW | MP | HMW | LMW |
| F1 | 98.2 | ND | 1.8 | 97.4 | ND | 2.6 | 97.5 | ND | 2.5 |
| F2 | 97.8 | ND | 2.2 | 97.9 | ND | 2.1 | 97.0 | ND | 3.0 |
| F3 | 97.9 | ND | 2.1 | 97.4 | ND | 2.6 | 96.7 | ND | 3.3 |
| F4 | 97.9 | ND | 2.1 | 97.6 | ND | 2.4 | 95.9 | ND | 4.1 |
| F5 | 98.2 | ND | 1.8 | 97.8 | ND | 2.2 | 96.3 | ND | 3.7 |
| F6 | 98.0 | ND | 2.0 | 97.9 | ND | 2.1 | 96.3 | ND | 3.7 |
| F7 | 97.4 | ND | 2.6 | 97.9 | ND | 2.1 | 96.5 | ND | 3.6 |
| F8 | 97.9 | ND | 2.1 | 97.6 | ND | 2.4 | 96.2 | ND | 3.8 |
| F9 | 97.8 | ND | 2.2 | 97.9 | ND | 2.1 | 96.1 | ND | 3.9 |
| F10 | 98.4 | ND | 1.6 | 97.8 | ND | 2.2 | 96.1 | ND | 3.9 |
| F11 | 96.7 | ND | 3.3 | 97.8 | ND | 2.2 | 95.2 | ND | 4.8 |
| F12 | 97.5 | ND | 2.5 | 98.3 | ND | 1.7 | 94.7 | ND | 5.3 |
| | 40° C. | | | | | | | | |
| Formula | 1 W | | | 2 W | | | 4 W | | |
| No. | MP | HMW | LMW | MP | HMW | LMW | MP | HMW | LMW |
| F1 | 97.6 | ND | 2.4 | 94.9 | ND | 5.1 | 91.3 | ND | 8.7 |
| F2 | 97.1 | ND | 2.9 | 96.3 | ND | 3.7 | 93.3 | ND | 6.8 |
| F3 | 96.5 | ND | 3.5 | 96.8 | ND | 3.2 | 92.7 | ND | 7.3 |
| F4 | 97.5 | ND | 2.5 | 95.0 | ND | 5.0 | 91.2 | ND | 8.8 |
| F5 | 97.3 | NL | 2.7 | 96.7 | ND | 3.3 | 92.1 | ND | 7.9 |
| F6 | 96.8 | ND | 3.2 | 96.7 | ND | 3.3 | 91.8 | ND | 8.3 |
| F7 | 97.0 | ND | 3.0 | 96.2 | ND | 3.8 | 92.9 | ND | 7.1 |
| F8 | 97.0 | ND | 3.0 | 97.1 | ND | 2.9 | 92.4 | ND | 7.6 |
| F9 | 97.1 | ND | 2.9 | 96.9 | ND | 3.1 | 91.2 | ND | 8.8 |
| F10 | 97.0 | ND | 3.0 | 96.2 | ND | 3.8 | 90.3 | ND | 9.7 |
| F11 | 96.1 | NL | 3.9 | 95.4 | ND | 4.6 | 87.0 | ND | 13.0 |
| F12 | 93.9 | NL | 6.1 | 94.2 | ND | 5.8 | 82.8 | ND | 17.2 |

Note:
MP: main peak;
HMW: high molecular weight, high polymer;
LMW: low molecular weight, oligomer;
ND: not detected

TABLE 15
CE-SDS-R (%) detection results for pH/buffer system screening

| | | | 25° C. | | | | 40° C. | | | | | |
|---|---|---|---|---|---|---|---|---|---|---|---|---|
| Formula | T0 | | 1 W | | 2 W | | 1 W | | 2 W | | 4 W | |
| No. | LC + HC | Other | LC + HC | Other | LC + HC | Other | LC + HC | Other | LC + HC | Other | LC + HC | Other |
| F1 | 97.6 | 2.4 | 97.5 | 2.5 | 96.9 | 3.1 | 97.2 | 2.8 | 94.9 | 5.1 | 92.9 | 7.1 |
| F2 | 98.0 | 2.0 | 97.6 | 2.5 | 96.8 | 3.2 | 97 | 3 | 95.7 | 4.3 | 93.6 | 6.4 |
| F3 | 97.8 | 2.2 | 97.6 | 2.4 | 97.2 | 2.8 | 97.2 | 2.8 | 95.4 | 4.6 | 94.4 | 5.6 |
| F4 | 98.1 | 2.0 | 97.4 | 2.7 | 96.8 | 3.2 | 96.5 | 3.5 | 95.2 | 4.9 | 92.2 | 7.8 |
| F5 | 98.0 | 2.0 | 97.5 | 2.5 | 97.2 | 2.8 | 97.2 | 2.8 | 96.2 | 3.8 | 93.6 | 6.4 |
| F6 | 98.1 | 1.9 | 97.7 | 2.3 | 97.2 | 2.8 | 95.3 | 4.8 | 96.7 | 3.3 | 93.8 | 6.2 |
| F7 | 98.7 | 1.3 | 97.6 | 2.4 | 97.1 | 2.9 | 97.2 | 2.9 | 96.0 | 4.0 | 94.0 | 6.0 |
| F8 | 97.7 | 2.3 | 97.3 | 2.7 | 97.1 | 2.9 | 97.0 | 3.1 | 96.0 | 4.0 | 95.3 | 4.7 |
| F9 | 98.6 | 1.4 | 97.4 | 2.6 | 97.3 | 2.7 | 96.5 | 3.5 | 95.8 | 4.2 | 94.9 | 5.1 |
| F10 | 98.0 | 2.0 | 97.3 | 2.7 | 97.2 | 2.8 | 94.9 | 5.1 | 94.9 | 5.1 | 93.4 | 6.6 |
| F11 | 98.1 | 1.9 | 97.0 | 3.0 | 97.2 | 2.9 | 94.2 | 5.8 | 93.8 | 6.2 | 91.4 | 8.6 |
| F12 | 98.0 | 2.0 | 97.0 | 3.0 | 96.3 | 3.7 | 92.8 | 7.2 | 91.8 | 8.3 | 87.0 | 13.0 |

Note:
LC + HC: light chain + heavy chain;
other: additional

TABLE 16
ELISA-Binding (%) detection results
for pH/buffer system screening

| Formula No. | T0 | 25° C.-4 W | 40° C.-4 W |
|---|---|---|---|
| F1 | 98 | 119 | 90 |
| F2 | 111 | 111 | 87 |
| F3 | 105 | 108 | 98 |
| F4 | 98 | 123 | 85 |
| F5 | 89 | 96 | 93 |
| F6 | 100 | 101 | 99 |
| F7 | 95 | 113 | 121 |
| F8 | 90 | 117 | 110 |
| F9 | 89 | 104 | 116 |
| F10 | 89 | 97 | 85 |
| F11 | 108 | 98 | 105 |
| F12 | 107 | 103 | 103 |

### 2.3 Analysis of Results

(1) Appearance: The appearance of the acetic acid system was better than that of the histidine, citric acid, and phosphate buffer systems.
(2) pH value: The pH of all buffer systems did not change significantly throughout the investigation.
(3) Protein concentration: The protein concentration of all buffer systems did not change significantly throughout the investigation.
(4) Thermal stability (DSF): There was no significant difference in Tm value at TO among all buffer systems.
(5) DLS: The particle size of the acetic acid buffer system was smaller than that of other buffer systems, where F1<F2<F3.
(6) iCIEF: The charge isomer main peak of the histidine buffer system and the acetic acid buffer system was significantly superior to that of the citric acid and phosphate systems.
(7) SE-HPLC: There was no significant difference in monomer purity between the acetic acid and histidine buffer systems. When the samples were stored at 40° C. for 4 W, the monomer purity of the citric acid and phosphate systems was significantly lower than that of the acetic acid and histidine buffer systems.
(8) CE-SDS (NR): When the samples were stored at 40° C. for 4 W, the F2 formula had the highest monomer main peak proportion and the highest protein purity.
(9) CE-SDS (R): When the proportion of the monomer main peaks was compared, the protein purity of the acetic acid and histidine buffer systems was superior to that of the citric acid and phosphate systems, while there was no significant difference between the acetic acid and histidine buffer systems.
(10) ELISA-Binding: There was no significant difference in binding activity among all buffer systems (note: the normal range of ELISA fluctuation was 70-130).

Summary: The comprehensive evaluation of the detection indicators of the 12 candidate formulas shows that the detection indexes of the acetic acid and histidine buffer systems are significantly superior to those of the citric acid and phosphate systems. The acetic acid buffer system is slightly superior to the histidine buffer system in DLS, CE-SDS (R), and other indicators, where the F2 formula (20 mM acetic acid-sodium acetate, pH 5.0) is good in appearance, particle size, and purity and slightly superior in stability, as compared to other formulas. Thus, the F2 formula was selected for subsequent excipient screening.

### Example 3. Excipient Screening

### 3.1 Study Protocol

(1) Based on the optimal pH/buffer system (20 mM acetic acid-sodium acetate pH 5.0) obtained by screening in Example 2, 6 different excipients were designed and added, and the specific information of the candidate formulas is shown in Table 12.
(2) The stability of the PVRIG/TIGIT bispecific antibody LC-BsAb-002 (with a protein concentration of 50 mg/mL) in 6 candidate formulas was comprehensively investigated through a stability experiment, an agitation experiment, and a freeze-thawing experiment to select the optimal excipient for the subsequent surfactant development. The assessment indicators included appearance, pH, protein concentration, dynamic light scattering (DLS), thermal stability (DSF), osmotic pressure, iCIEF, SE-HPLC, CE-SDS (NR&R), and binding activity (ELISA-Binding). The investigation protocol is detailed in Table 17.

TABLE 17
Formulas and investigation protocol for excipient screening

| | Formulation | | | | | | | | | | | | | | |
|---|---|---|---|---|---|---|---|---|---|---|---|---|---|---|---|
| Formula | pH/buffer | | | 2~8° C. | | 25° C. | | | 40° C. | | | Agitation | | F/T^{a} | |
| No. | system | Excipient | T0 | 4 W | 3 M | 2 W | 4 W | 3 M | 1 W | 2 W | 4 W | 1 D | 3 D | 3 C | 5 C |
| F2-1 | 20 mM acetic | 2% glycine* | X, Y, | X, Y | X, Y | X | X, Y | X, Y | X | X | X, Y | X | X, Y | X | X, Y |
| F2-2 | acid-sodium | 140 mM | Z | | | | | | | | | | | | |
| | acetate pH | arginine | | | | | | | | | | | | | |
| | 5.0, 0.04% | hydrochloride | | | | | | | | | | | | | |
| F2-3 | PS80 | 140 mM | | | | | | | | | | | | | |
| | | sodium | | | | | | | | | | | | | |
| | | chloride | | | | | | | | | | | | | |
| F2-4 | | 8% | | | | | | | | | | | | | |
| | | sucrose* | | | | | | | | | | | | | |
| F2-5 | | 8% | | | | | | | | | | | | | |
| | | trehalose* | | | | | | | | | | | | | |
| F2-6 | | 4.5% | | | | | | | | | | | | | |
| | | sorbitol* | | | | | | | | | | | | | |

Note:
X = appearance, pH, protein concentration, DLS, iCIEF, SE-HPLC, CE-SDS (NR&R);
Y = ELISA-Binding, insoluble particle (MFI);
Z = DSF, osmotic pressure.
*All % in the table refers to % (w/v).
^{a}F/T: freeze/thaw. M = month, D = day, C = round, the same below.

### 3.2 Screening Results

The major results of the excipient screening are summarized in Tables 18-32 below:

TABLE 18
Appearance detection results for excipient screening

| Formula | | 2~8° C. | | 25° C. | | | 40° C. |
|---|---|---|---|---|---|---|---|
| No. | T0 | 4 W | 3 M | 2 W | 4 W | 3 M | 1 W |
| F2-1 | Colorless, | Colorless, | Colorless, | Colorless, | Colorless, | Colorless, | Colorless, |
| | clear, no | clear, no | clear, no | clear, no | clear, no | clear, no | clear, no |
| | visible | visible | visible | visible | visible | visible | visible |
| | particles | particles | particles | particles | particles | particles | particles |
| F2-2 | Colorless, | Colorless, | Colorless, | Colorless, | Colorless, | Colorless, | Colorless, |
| | strongly | strongly | strongly | strongly | strongly | strongly | strongly |
| | opalescent, | opalescent, | opalescent, | opalescent, | opalescent, | opalescent, | opalescent, |
| | no visible | no visible | no visible | no visible | no visible | no visible | no visible |
| | particles | particles | particles | particles | particles | particles | particles |
| F2-3 | Colorless, | Colorless, | Colorless, | Colorless, | Colorless, | Colorless, | Colorless, |
| | strongly | strongly | strongly | strongly | strongly | strongly | strongly |
| | opalescent, | opalescent, | opalescent, | opalescent, | opalescent, | opalescent, | opalescent, |
| | no visible | no visible | no visible | no visible | no visible | no visible | no visible |
| | particles | particles | particles | particles | particles | particles | particles |
| F2-4 | Colorless, | Colorless, | Colorless, | Colorless, | Colorless, | Colorless, | Colorless, |
| | clear, no | clear, no | clear, no | clear, no | clear, no | clear, no | clear, no |
| | visible | visible | visible | visible | visible | visible | visible |
| | particles | particles | particles | particles | particles | particles | particles |
| F2-5 | Colorless, | Colorless, | Colorless, | Colorless, | Colorless, | Colorless, | Colorless, |
| | clear, no | clear, no | clear, no | clear, no | clear, no | clear, no | clear, no |
| | visible | visible | visible | visible | visible | visible | visible |
| | particles | particles | particles | particles | particles | particles | particles |
| F2-6 | Colorless, | Colorless, | Colorless, | Colorless, | Colorless, | Colorless, | Colorless, |
| | clear, no | clear, no | clear, no | clear, no | clear, no | clear, no | clear, no |
| | visible | visible | visible | visible | visible | visible | visible |
| | particles | particles | particles | particles | particles | particles | particles |

| Formula | 40° C. | | Agitation | | F/T | |
|---|---|---|---|---|---|---|
| No. | 2 W | 4 W | 1 D | 3 D | 3C | 5C |
| F2-1 | Colorless, | Colorless, | Colorless, | Colorless, | Colorless, | Colorless, |
| | clear, no | clear, no | clear, no | clear, no | clear, no | clear, no |
| | visible | visible | visible | visible | visible | visible |
| | particles | particles | particles | particles | particles | particles |
| F2-2 | Colorless, | Colorless, | Colorless, | Colorless, | Colorless, | Colorless, |
| | strongly | strongly | strongly | strongly | strongly | strongly |
| | opalescent, | opalescent, | opalescent, | opalescent, | opalescent, | opalescent, |
| | no visible | no visible | no visible | no visible | no visible | no visible |
| | particles | particles | particles | particles | particles | particles |
| F2-3 | Colorless, | Colorless, | Colorless, | Colorless, | Colorless, | Colorless, |
| | strongly | strongly | strongly | strongly | strongly | strongly |
| | opalescent, | opalescent, | opalescent, | opalescent, | opalescent, | opalescent, |
| | no visible | no visible | no visible | no visible | no visible | no visible |
| | particles | particles | particles | particles | particles | particles |
| F2-4 | Colorless, | Colorless, | Colorless, | Colorless, | Colorless, | Colorless, |
| | clear, no | clear, no | clear, no | clear, no | clear, no | clear, no |
| | visible | visible | visible | visible | visible | visible |
| | particles | particles | particles | particles | particles | particles |
| F2-5 | Colorless, | Colorless, | Colorless, | Colorless, | Colorless, | Colorless, |
| | clear, no | clear, no | clear, no | clear, no | clear, no | clear, no |
| | visible | visible | visible | visible | visible | visible |
| | particles | particles | particles | particles | particles | particles |
| F2-6 | Colorless, | Colorless, | Colorless, | Colorless, | Colorless, | Colorless, |
| | clear, no | clear, no | clear, no | clear, no | clear, no | clear, no |
| | visible | visible | visible | visible | visible | visible |
| | particles | particles | particles | particles | particles | particles |

TABLE 19
pH detection results for excipient screening

| Formula | | 2~8° C. | | 25° C. | | | 40° C. | | | Agitation | | F/T | |
|---|---|---|---|---|---|---|---|---|---|---|---|---|---|
| No. | T0 | 4 W | 3 M | 2 W | 4 W | 3 M | 1 W | 2 W | 4 W | 1 D | 3 D | 3C | 5C |
| F2-1 | 5.2 | 5.2 | 5.2 | 5.2 | 5.2 | 5.2 | 5.2 | 5.2 | 5.2 | 5.2 | 5.2 | 5.2 | 5.2 |
| F2-2 | 5.1 | 5.1 | 5.1 | 5.1 | 5.1 | 5.2 | 5.1 | 5.2 | 5.1 | 5.2 | 5.2 | 5.1 | 5.1 |
| F2-3 | 5.2 | 5.1 | 5.2 | 5.2 | 5.2 | 5.2 | 5.2 | 5.2 | 5.2 | 5.2 | 5.2 | 5.2 | 5.2 |
| F2-4 | 5.2 | 5.2 | 5.2 | 5.1 | 5.2 | 5.2 | 5.2 | 5.2 | 5.2 | 5.2 | 5.2 | 5.1 | 5.2 |
| F2-5 | 5.2 | 5.2 | 5.1 | 5.1 | 5.2 | 5.2 | 5.1 | 5.2 | 5.2 | 5.2 | 5.2 | 5.1 | 5.2 |
| F2-6 | 5.1 | 5.1 | 5.2 | 5.1 | 5.2 | 5.2 | 5.2 | 5.2 | 5.2 | 5.2 | 5.2 | 5.1 | 5.2 |

TABLE 20
Protein concentration (mg/mL) detection results for excipient screening

| Formula | | 2~8° C. | | 25° C. | | | 40° C. | | | Agitation | | F/T | |
|---|---|---|---|---|---|---|---|---|---|---|---|---|---|
| No. | T0 | 4 W | 3 M | 2 W | 4 W | 3 M | 1 W | 2 W | 4 W | 1 D | 3 D | 3C | 5C |
| F2-1 | 50.4 | 50.4 | 50.7 | 50.4 | 50.6 | 50.4 | 50.4 | 50.6 | 50.5 | 50.4 | 50.3 | 50.5 | 50.3 |
| F2-2 | 50.6 | 50.5 | 50.5 | 50.6 | 50.6 | 50.8 | 50.7 | 50.6 | 50.6 | 50.5 | 50.8 | 50.7 | 50.6 |
| F2-3 | 49.9 | 50.3 | 50.4 | 50.7 | 50.3 | 50.3 | 50.3 | 50.4 | 50.3 | 50.3 | 50.4 | 50.5 | 50.3 |
| F2-4 | 51.7 | 52.0 | 52.4 | 51.9 | 52.0 | 51.8 | 51.8 | 51.9 | 51.9 | 51.6 | 51.7 | 51.7 | 51.7 |
| F2-5 | 51.4 | 51.7 | 51.5 | 51.9 | 51.5 | 51.6 | 51.5 | 52.0 | 51.6 | 51.3 | 51.4 | 51.5 | 51.6 |
| F2-6 | 50.7 | 50.7 | 50.7 | 50.8 | 51.0 | 50.7 | 50.6 | 50.8 | 50.9 | 50.5 | 50.4 | 50.6 | 50.5 |

TABLE 21
DLS (diameter, nm) detection results for excipient screening

| Formula | | 2~8° C. | | 25° C. | | | 40° C. | | | Agitation | | F/T | |
|---|---|---|---|---|---|---|---|---|---|---|---|---|---|
| No. | T0 | 4 W | 3 M | 2 W | 4 W | 3 M | 1 W | 2 W | 4 W | 1 D | 3 D | 3C | 5C |
| F2-1 | 8.4 | 8.1 | 8.0 | 8.2 | 8.2 | 8.0 | 8.3 | 8.5 | 9.5 | 7.7 | 8.2 | 9.5 | 8.9 |
| F2-2 | 18.1 | 16.6 | 16.3 | 17.5 | 16.8 | 17.0 | 17.5 | 18.5 | 16.3 | 16.1 | 18.3 | 17.8 | 17.1 |
| F2-3 | 23.9 | 21.1 | 20.8 | 23.1 | 21.3 | 21.7 | 23.0 | 25.2 | 23.2 | 23.1 | 24.2 | 23.6 | 22.9 |
| F2-4 | 9.2 | 9.5 | 9.5 | 9.4 | 9.4 | 9.5 | 9.6 | 10.0 | 9.8 | 9.5 | 9.9 | 9.6 | 9.3 |
| F2-5 | 9.9 | 9.1 | 9.1 | 9.3 | 8.7 | 9.4 | 8.8 | 9.9 | 9.8 | 9.4 | 9.8 | 9.4 | 9.3 |
| F2-6 | 9.0 | 8.4 | 8.3 | 8.6 | 7.9 | 8.1 | 8.1 | 8.5 | 9.0 | 8.8 | 9.1 | 8.6 | 8.5 |

TABLE 22
Insoluble particle (MFI, particles/mL) detection results for excipient screening

| | | | 2~8° C. | | | | 25° C. | | | | 40° C. | | Agitation | | F/T | |
|---|---|---|---|---|---|---|---|---|---|---|---|---|---|---|---|---|
| Formula | T0 | | 4 W | | 3 M | | 4 W | | 3 M | | 4 W | | 3 D | | 5C | |
| No. | ≥10* | ≥25* | ≥10 | ≥25 | ≥10 | ≥25 | ≥10 | ≥25 | ≥10 | ≥25 | ≥10 | ≥25 | ≥10 | ≥25 | ≥10 | ≥25 |
| F2-1 | 2 | 0 | 21 | 0 | 59 | 0 | 75 | 3 | 25 | 3 | 64 | 5 | 128 | 16 | 35 | 0 |
| F2-2 | 4 | 0 | 35 | 7 | — | — | 37 | 0 | 30 | 7 | 7 | 0 | 10 | 0 | 0 | 0 |
| F2-3 | 2 | 0 | 89 | 5 | 19 | 3 | 162 | 19 | 44 | 5 | 148 | 10 | 14 | 0 | 50 | 7 |
| F2-4 | 13 | 4 | 16 | 0 | — | — | 35 | 5 | 3 | 0 | 10 | 0 | 10 | 3 | 7 | 0 |
| F2-5 | 88 | 0 | 5 | 0 | — | — | 19 | 0 | 16 | 3 | 7 | 0 | 21 | 0 | 10 | 0 |
| F2-6 | 51 | 2 | 59 | 5 | 10 | 5 | 32 | 3 | 16 | 10 | 10 | 0 | 7 | 0 | 87 | 7 |

≥10: particles with the diameter ≥10 μm; ≥25: particles with the diameter ≥25 μm.

TABLE 23
DSF (° C.) and osmotic pressure (mOsm/kg) detection
results at T0 sampling point for excipient screening

| | T0 | | |
|---|---|---|---|
| Formula No. | Tm1 | Tm2 | Osmotic pressure |
| F2-1 | 68.9 | 78.1 | 0.321 |
| F2-2 | 68.8 | 77.7 | 0.295 |
| F2-3 | 68.8 | 77.7 | 0.315 |
| F2-4 | 68.6 | 77.5 | 0.331 |
| F2-5 | 69.1 | 78.0 | 0.305 |
| F2-6 | 68.9 | 78.0 | 0.321 |

TABLE 24
iCIEF (%) detection results of stability experiment at 2-8° C. and 25° C. for excipient screening

| | | | | 2~8° C. | | | | | | 25° C. | | | | | | | | |
|---|---|---|---|---|---|---|---|---|---|---|---|---|---|---|---|---|---|---|
| Formula | T0 | | | 4 W | | | 3 M | | | 2 W | | | 4 W | | | 3 M | | |
| No. | MP | AP | BP | MP | AP | BP | MP | AP | BP | MP | AP | BP | MP | AP | BP | MP | AP | BP |
| F2-1 | 58.9 | 36.6 | 4.5 | 58.2 | 36.3 | 5.7 | 58.9 | 34.8 | 6.3 | 54.7 | 37.5 | 7.8 | 52.9 | 37.1 | 10 | 40.4 | 44.7 | 14.9 |
| F2-2 | 64.1 | 32.1 | 3.7 | 58.2 | 36.1 | 5.8 | 57.2 | 36.0 | 6.9 | 55.6 | 36.8 | 7.6 | 54.7 | 34 | 11.3 | 45.4 | 35.5 | 19.1 |
| F2-3 | 61.4 | 34.7 | 3.9 | 59.2 | 35.5 | 5.5 | 57.4 | 35.8 | 6.7 | 54.9 | 37.5 | 7.7 | 53.4 | 35.3 | 11.3 | 45.5 | 36.0 | 18.6 |
| F2-4 | 58.4 | 36.2 | 5.5 | 58.1 | 36.5 | 5.5 | 57.1 | 36.2 | 6.6 | 54.4 | 37.4 | 8.3 | 52.4 | 36.7 | 10.8 | 45.1 | 37.7 | 17.3 |
| F2-5 | 55.8 | 39.3 | 4.9 | 58 | 36.4 | 5.7 | 57.2 | 36.2 | 6.7 | 54.1 | 38.2 | 7.7 | 52.4 | 36.8 | 10.8 | 45.8 | 37.6 | 16.6 |
| F2-6 | 57.1 | 37.9 | 5 | 58.3 | 35.9 | 5.8 | 58.7 | 34.9 | 6.4 | 54.2 | 37.5 | 8.3 | 54 | 35 | 10.9 | 44.9 | 37.7 | 17.3 |

TABLE 25
iCIEF (%) detection results of stability experiment at 40° C., agitation
experiment, and freeze-thawing experiment for excipient screening

| | | | | 40° C. | | | | | | | | |
|---|---|---|---|---|---|---|---|---|---|---|---|---|
| Formula | T0 | | | 1 W | | | 2 W | | | 4 W | | |
| No. | MP | AP | BP | MP | AP | BP | MP | AP | BP | MP | AP | BP |
| F2-1 | 58.9 | 36.6 | 4.5 | 49 | 38.5 | 12.6 | 36.1 | 47.1 | 16.7 | 22.5 | 53 | 24.5 |
| F2-2 | 64.1 | 32.1 | 3.7 | 51.2 | 34.8 | 14.1 | 38.2 | 40.9 | 21 | 28 | 41.5 | 30.4 |
| F2-3 | 61.4 | 34.7 | 3.9 | 51.7 | 34.2 | 14.1 | 36.7 | 41.1 | 22.2 | 27.7 | 42.6 | 29.8 |
| F2-4 | 58.4 | 36.2 | 5.5 | 49.6 | 36.6 | 13.7 | 37.6 | 44.2 | 18.4 | 27.5 | 45.8 | 26.6 |
| F2-5 | 55.8 | 39.3 | 4.9 | 50.7 | 36.1 | 13.3 | 38.2 | 43.8 | 18 | 27.6 | 45.8 | 26.6 |
| F2-6 | 57.1 | 37.9 | 5 | 50.7 | 36.4 | 12.9 | 38 | 43.9 | 18.2 | 27.9 | 45.3 | 26.8 |

| | | | | Agitation | | | | | | F/T | | | | | |
|---|---|---|---|---|---|---|---|---|---|---|---|---|---|---|---|
| Formula | T0 | | | 1 D | | | 3 D | | | 3C | | | 5C | | |
| No. | MP | AP | BP | MP | AP | BP | MP | AP | BP | MI | AP | BP | MP | AP | BP |
| F2-1 | 58.9 | 36.6 | 4.5 | 52 | 42.9 | 5.3 | 53.9 | 41.1 | 5.1 | 60.2 | 34.9 | 4.8 | 58.9 | 36.7 | 4.5 |
| F2-2 | 64.1 | 32.1 | 3.7 | 61.5 | 32.9 | 5.6 | 54.8 | 39.2 | 5.6 | 58.7 | 35.9 | 5.3 | 60 | 34.7 | 5.2 |
| F2-3 | 61.4 | 34.7 | 3.9 | 59.4 | 35 | 5.6 | 59.3 | 36.7 | 4 | 59.9 | 35 | 5.2 | 60.8 | 34.7 | 4.4 |
| F2-4 | 58.4 | 36.2 | 5.5 | 59.1 | 35.5 | 5.4 | 53.7 | 41.1 | 5.1 | 60.1 | 35.3 | 4.7 | 60.2 | 34.7 | 5 |
| F2-5 | 55.8 | 39.3 | 4.9 | 51.2 | 44.2 | 4.6 | 55.9 | 38.9 | 5.2 | 60.5 | 34.6 | 5 | 60.2 | 34.8 | 4.9 |
| F2-6 | 57.1 | 37.9 | 5 | 58.5 | 36.2 | 5.2 | 54.8 | 40 | 5.2 | 60.7 | 34.4 | 5 | 60.3 | 34.9 | 4.9 |

Note:
MP: main peak;
AP: acidic peak;
BP: basic peak

TABLE 26
SE-HPLC (%) detection results of stability experiment at 2-
8° C. and 25° C. for excipient screening

| | | | | 2~8° C. | | | | | |
|---|---|---|---|---|---|---|---|---|---|
| Formula | T0 | | | 4 W | | | 3 M | | |
| No. | MP | HMW | LMW | MP | HMW | LMW | MP | HMW | LMW |
| F2-1 | 98.7 | 1.3 | ND | 98.9 | 1.1 | ND | 98.6 | 1.4 | ND |
| F2-2 | 98.7 | 1.3 | ND | 98.9 | 1.1 | ND | 98.6 | 1.4 | ND |
| F2-3 | 98 | 2 | ND | 98.5 | 1.5 | ND | 97.9 | 2.1 | ND |
| F2-4 | 98.7 | 1.3 | ND | 98.8 | 1.2 | ND | 98.6 | 1.4 | ND |
| F2-5 | 98.6 | 1.4 | ND | 98.9 | 1.1 | ND | 98.6 | 1.4 | ND |
| F2-6 | 98.7 | 1.3 | ND | 98.9 | 1.1 | ND | 98.6 | 1.4 | ND |
| | 25° C. | | | | | | | | |
| Formula | 2 W | | | 4 W | | | 3 M | | |
| No. | MP | HMW | LMW | MP | HMW | LMW | MP | HMW | LMW |
| F2-1 | 98.7 | 1.3 | ND | 98.3 | 1.6 | 0.1 | 95.2 | 2.9 | 1.9 |
| F2-2 | 98.8 | 1.2 | ND | 98.3 | 1.5 | 0.2 | 95.3 | 2.9 | 1.8 |
| F2-3 | 98.2 | 1.8 | ND | 97.7 | 2.2 | 0.2 | 94.2 | 4 | 1.8 |
| F2-4 | 98.8 | 1.2 | ND | 98.4 | 1.5 | 0.1 | 96 | 2.3 | 1.7 |
| F2-5 | 98.8 | 1.2 | ND | 98.4 | 1.5 | 0.1 | 96.2 | 2.2 | 1.6 |
| F2-6 | 98.8 | 1.2 | ND | 98.4 | 1.5 | 0.1 | 96.1 | 2.2 | 1.7 |

TABLE 27
SE-HPLC (%) detection results of stability experiment at 40° C., agitation experiment, and freeze-thawing experiment for excipient screening

| | | | | 40° C. | | | | | | | | |
|---|---|---|---|---|---|---|---|---|---|---|---|---|
| Formula | T0 | | | 1 W | | | 2 W | | | 4 W | | |
| No. | MP | HMW | LMW | MP | HMW | LMW | MP | HMW | LMW | MP | HMW | LMW |
| F2-1 | 98.7 | 1.3 | ND | 96.7 | 3.1 | 0.2 | 96.9 | 3 | 0.2 | 92 | 5 | 2.9 |
| F2-2 | 98.7 | 1.3 | ND | 97.5 | 2.3 | 0.2 | 95.9 | 3.8 | 0.3 | 90.7 | 6 | 3.2 |
| F2-3 | 98 | 2 | ND | 96.8 | 3 | 0.2 | 95.1 | 4.6 | 0.3 | 89.2 | 7.4 | 3.4 |
| F2-4 | 98.7 | 1.3 | ND | 98.1 | 1.7 | 0.2 | 97.7 | 2.2 | 0.2 | 93.9 | 3.5 | 2.6 |
| F2-5 | 98.6 | 1.4 | NL | 98.1 | 1.7 | 0.2 | 97.6 | 2.2 | 0.2 | 94.1 | 3.3 | 2.5 |
| F2-6 | 98.7 | 1.3 | ND | 98 | 1.8 | 0.2 | 97.6 | 2.3 | 0.2 | 93.9 | 3.5 | 2.6 |

| | | | | Agitation | | | | | | F/T | | | | | |
|---|---|---|---|---|---|---|---|---|---|---|---|---|---|---|---|
| Formula | T0 | | | 1 D | | | 3 D | | | 3C | | | 5C | | |
| No. | MP | HMW | LMW | MP | HMW | LMW | MP | HMW | LMW | MP | HMW | LMW | MP | HMW | LMW |
| F2-1 | 98.7 | 1.3 | ND | 97.5 | 2.4 | 0.1 | 98.7 | 1.3 | 0 | 95.1 | 4.8 | ND | 94.1 | 5.9 | ND |
| F2-2 | 98.7 | 1.3 | ND | 98.6 | 1.3 | 0.1 | 97.4 | 2.6 | 0 | 98.6 | 1.4 | ND | 98.6 | 1.4 | ND |
| F2-3 | 98 | 2 | ND | 98.7 | 1.3 | 0 | 98.3 | 1.7 | 0 | 98.3 | 1.7 | ND | 98.2 | 1.8 | ND |
| F2-4 | 98.7 | 1.3 | ND | 98.7 | 1.3 | 0 | 98.6 | 1.3 | 0 | 98.7 | 1.3 | ND | 98.6 | 1.4 | 0 |
| F2-5 | 98.6 | 1.4 | ND | 98.6 | 1.3 | 0 | 98.5 | 1.4 | 0 | 98.7 | 1.3 | NL | 98.6 | 1.4 | ND |
| F2-6 | 98.7 | 1.3 | ND | 98.4 | 1.6 | 0 | 98.6 | 1.4 | 0 | 98.7 | 1.3 | ND | 98.5 | 1.4 | 0.1 |

Note:
MP: main peak;
HMW: high molecular weight, high polymer;
LMW: low molecular weight, oligomer;
ND: not detected

TABLE 28
CE-SDS-NR (%) detection results of stability experiment at
2-8° C. and 25° C. for excipient screening

| | | | | 2~8° C. | | | | | |
|---|---|---|---|---|---|---|---|---|---|
| Formula | T0 | | | 4 W | | | 3 M | | |
| No. | MP | HMW | LMW | MP | HMW | LMW | MP | HMW | LMW |
| F2-1 | 96.8 | ND | 3.1 | 96.2 | ND | 3.8 | 95.0 | ND | 5.0 |
| F2-2 | 95.3 | ND | 4.7 | 96.4 | ND | 3.6 | 95.2 | ND | 4.8 |
| F2-3 | 94.4 | ND | 5.7 | 96.2 | ND | 3.8 | 95.6 | ND | 4.4 |
| F2-4 | 97.0 | ND | 3.0 | 95.4 | ND | 4.6 | 95.7 | ND | 4.3 |
| F2-5 | 96.7 | ND | 3.3 | 96.0 | ND | 4.0 | 95.8 | ND | 4.2 |
| F2-6 | 96.3 | ND | 3.7 | 96.3 | ND | 3.7 | 96.1 | ND | 3.9 |
| | 25° C. | | | | | | | | |
| Formula | 2 W | | | 4 W | | | 3 M | | |
| No. | MP | HMW | LMW | MP | HMW | LMW | MP | HMW | LMW |
| F2-1 | 96.9 | ND | 3.1 | 96.1 | ND | 3.9 | 94.2 | ND | 5.8 |
| F2-2 | 96.8 | ND | 3.2 | 96.1 | ND | 4.0 | 94.6 | ND | 5.5 |
| F2-3 | 97.1 | ND | 3.0 | 95.9 | ND | 4.2 | 95.4 | ND | 4.6 |
| F2-4 | 95.2 | ND | 4.8 | 95.8 | ND | 4.2 | 94.5 | ND | 5.5 |
| F2-5 | 96.1 | ND | 3.9 | 96.3 | ND | 3.7 | 95.1 | ND | 4.9 |
| F2-6 | 96.3 | ND | 3.7 | 96.9 | ND | 3.1 | 95.0 | ND | 5.0 |

TABLE 29
CE-SDS-NR (%) detection results of stability experiment at 40° C., agitation experiment, and freeze-thawing experiment for excipient screening

| | | | | 40° C. | | | | | | | | |
|---|---|---|---|---|---|---|---|---|---|---|---|---|
| Formula | T0 | | | 1 W | | | 2 W | | | 4 W | | |
| No. | MP | HMW | LMW | MP | HMW | LMW | MP | HMW | LMW | MP | HMW | LMW |
| F2-1 | 96.8 | ND | 3.1 | 95.5 | ND | 4.5 | 95.1 | ND | 4.9 | 93.2 | ND | 6.8 |
| F2-2 | 95.3 | ND | 4.7 | 95.2 | ND | 4.8 | 95.9 | ND | 4.2 | 93.1 | ND | 6.9 |
| F2-3 | 94.4 | ND | 5.7 | 95.4 | ND | 4.6 | 95.5 | ND | 4.5 | 93.8 | ND | 6.2 |
| F2-4 | 97.0 | ND | 3.0 | 96.4 | NL | 3.6 | 95.3 | ND | 4.7 | 94.4 | ND | 5.7 |
| F2-5 | 96.7 | ND | 3.3 | 96.0 | ND | 4.1 | 94.9 | ND | 5.1 | 93.4 | ND | 6.6 |
| F2-6 | 96.3 | NL | 3.7 | 96.2 | ND | 3.8 | 95.7 | ND | 4.3 | 94.2 | ND | 5.8 |

| | | | | Agitation | | | | | | F/T | | | | | |
|---|---|---|---|---|---|---|---|---|---|---|---|---|---|---|---|
| Formula | T0 | | | 1 D | | | 3 D | | | 3C | | | 5C | | |
| No. | MP | HMW | LMW | MP | HMW | LMW | MP | HMW | LMW | MP | HMW | LMW | MP | HMW | LMW |
| F2-1 | 96.8 | ND | 3.1 | 93.7 | ND | 6.4 | 97.3 | ND | 2.7 | 95.8 | ND | 4.3 | 95.8 | ND | 4.2 |
| F2-2 | 95.3 | ND | 4.7 | 94.1 | ND | 5.9 | 97.0 | ND | 2.9 | 94.9 | 0 | 5.1 | 95.9 | ND | 4.1 |
| F2-3 | 94.4 | ND | 5.7 | 95.3 | ND | 4.7 | 96.6 | ND | 3.4 | 95.7 | ND | 4.3 | 96.0 | ND | 4.0 |
| F2-4 | 97.0 | ND | 3.0 | 97.1 | ND | 2.9 | 96.6 | ND | 3.4 | 95.8 | ND | 4.2 | 96.0 | ND | 4.0 |
| F2-5 | 96.7 | ND | 3.3 | 96.6 | ND | 3.4 | 96.1 | ND | 3.9 | 96.5 | ND | 3.5 | 96.6 | ND | 3.5 |
| F2-6 | 96.3 | ND | 3.7 | 95.2 | ND | 4.8 | 96.8 | ND | 3.2 | 96.4 | ND | 3.6 | 96.2 | ND | 3.9 |

Note:
MP: main peak;
HMW: high molecular weight, high polymer;
LMW: low molecular weight, oligomer;
ND: not detected

TABLE 30
CE-SDS-R (%) detection results of stability experiment at 2-8° C. and 25° C. for excipient screening

| | | | 2~8° C. | | | | 25° C. | | | | | |
|---|---|---|---|---|---|---|---|---|---|---|---|---|
| Formula | T0 | | 4 W | | 3 M | | 2 W | | 4 W | | 3 M | |
| No. | LC + HC | Other | LC + HC | Other | LC + HC | Other | LC + HC | Other | LC + HC | Other | LC + HC | Other |
| F2-1 | 99.4 | 0.7 | 98.8 | 1.2 | 98.5 | 1.5 | 98.7 | 1.3 | 98.3 | 1.7 | 97.5 | 2.5 |
| F2-2 | 99.6 | 0.4 | 98.8 | 1.3 | 98.4 | 1.6 | 99.0 | 1.0 | 98.7 | 1.4 | 97.6 | 2.4 |
| F2-3 | 99.5 | 0.5 | 98.3 | 1.7 | 98.5 | 1.5 | 98.9 | 1.1 | 98.6 | 1.4 | 97.4 | 2.6 |
| F2-4 | 98.2 | 1.8 | 98.1 | 2.0 | 98.0 | 2.0 | 98.1 | 1.9 | 97.3 | 2.7 | 96.4 | 3.6 |
| F2-5 | 97.9 | 2.1 | 97.9 | 2.1 | 97.2 | 2.8 | 97.6 | 2.4 | 95.2 | 4.8 | 95.8 | 4.2 |
| F2-6 | 97.6 | 2.4 | 97.5 | 2.5 | 96.8 | 3.2 | 97.6 | 2.4 | 96.9 | 3.1 | 95.6 | 4.4 |

TABLE 31
CE-SDS-R (%) detection results of stability experiment at 40° C.,
agitation experiment, and freeze-thawing experiment for excipient screening

| | | | 40° C. | | | | | |
|---|---|---|---|---|---|---|---|---|
| Formula | T0 | | 1 W | | 2 W | | 4 W | |
| No. | LC + HC | Other | LC + HC | Other | LC + HC | Other | LC + HC | Other |
| F2-1 | 99.4 | 0.7 | 98.8 | 1.2 | 98.4 | 1.6 | 96.0 | 4.0 |
| F2-2 | 99.6 | 0.4 | 99.0 | 1.0 | 98.1 | 1.9 | 96.6 | 3.4 |
| F2-3 | 99.5 | 0.5 | 99.0 | 1.0 | 98.8 | 1.2 | 96.6 | 3.4 |
| F2-4 | 98.2 | 1.8 | 97.5 | 2.5 | 97.0 | 3.0 | 97.0 | 3.1 |
| F2-5 | 97.9 | 2.1 | 97.1 | 2.9 | 96.8 | 3.2 | 94.2 | 5.8 |
| F2-6 | 97.6 | 2.4 | 97.4 | 2.6 | 96.7 | 3.3 | 94.9 | 5.1 |

| | | | Agitation | | | | F/T | | | |
|---|---|---|---|---|---|---|---|---|---|---|
| Formula | T0 | | 1 D | | 3 D | | 3C | | 5C | |
| No. | LC + HC | Other | LC + HC | Other | LC + HC | Other | LC + HC | Other | LC + HC | Other |
| F2-1 | 99.4 | 0.7 | 99.4 | 0.7 | 99.3 | 0.7 | 99.3 | 0.7 | 99.3 | 0.7 |
| F2-2 | 99.6 | 0.4 | 99.4 | 0.6 | 99.2 | 0.8 | 99.1 | 0.9 | 99.3 | 0.7 |
| F2-3 | 99.5 | 0.5 | 99.3 | 0.7 | 99.0 | 1.0 | 99.3 | 0.7 | 99.3 | 0.7 |
| F2-4 | 98.2 | 1.8 | 98.0 | 2.0 | 98.0 | 2.0 | 97.4 | 2.6 | 97.3 | 2.7 |
| F2-5 | 97.9 | 2.1 | 97.4 | 2.7 | 97.3 | 2.7 | 97.5 | 2.5 | 96.5 | 3.5 |
| F2-6 | 97.6 | 2.4 | 97.5 | 2.5 | 97.5 | 2.5 | 97.5 | 2.5 | 96.9 | 3.1 |

Note:
LC + HC: light chain + heavy chain;
other: additional

TABLE 32
ELISA-Binding (%) detection results for excipient screening

| Formula | | 2~8° C. | | 25° C. | | 40° C. | Agitation | F/T |
|---|---|---|---|---|---|---|---|---|
| No. | T0 | 4 W | 3 M | 4 W | 3 M | 4 W | 3 D | 5C |
| F2-1 | 96.8 | 89.7 | 88.4 | 108.2 | 87.5 | 90.0 | 98.2 | 85.7 |
| F2-2 | 105.8 | 92.9 | 87.2 | 110.7 | 86.2 | 80.1 | 101.9 | 87.9 |
| F2-3 | 100.1 | 88.2 | 106.2 | 102.3 | 95.8 | 85.4 | 110.9 | 91.6 |
| F2-4 | 98.8 | 93.8 | 97.8 | 106.4 | 85.5 | 93.8 | 95.9 | 90.3 |
| F2-5 | 101.8 | 94.9 | 87.5 | 91.2 | 88.0 | 86.9 | 99.7 | 94.6 |
| F2-6 | 108.4 | 108.6 | 97.6 | 101.8 | 88.5 | 90.2 | 105.4 | 97.2 |

### 3.3 Analysis of Results

(1) Appearance: The appearance of the arginine (F2-2) and sodium chloride (F2-3) excipient preparation formulas was significantly inferior to that of other excipient preparation formulas, and there was no significant difference in appearance among glycine (F2-1), sucrose (F2-4), trehalose (F2-5), and sorbitol (F2-6).
(2) pH value: There was no significant difference in pH among all candidate formulas throughout the investigation.
(3) Protein concentration: The protein concentration of all candidate formulas did not change significantly throughout the investigation.
(4) Thermal stability (DSF): There was no significant difference in Tm value at TO among all candidate formulas.
(5) DLS: The particle size of the arginine (F2-2) and sodium chloride (F2-3) excipient preparation formulas was greater than that of other excipient preparation formulas, and there was no significant difference in particle size among glycine (F2-1), sucrose (F2-4), trehalose (F2-5), and sorbitol (F2-6).
(6) Insoluble particle: The sucrose (F2-4) preparation formula was slightly better than other excipient preparation formulas in controlling the generation of insoluble particles.
(7) iCIEF: By combining all the investigation conditions, the charge isomer main peak of the glycine (F2-1) excipient preparation formula was the lowest (e.g., stored at 40° C. for 4 W and 25° C. for 4 W), and there was no significant difference among other excipient preparation formulas.
(8) SE-HPLC: There was no significant difference in monomer purity among the sucrose (F2-4), trehalose (F2-5), and sorbitol (F2-6) excipient preparation formulas. The monomer purity of the formulas F2-4 to F2-6 was significantly higher than that of the formulas F2-1 to F2-3 when stored at a high temperature for a long time.
(9) CE-SDS (NR): There was no significant difference in protein purity among all candidate formulas.
(10) CE-SDS (R): The sucrose (F2-4) excipient preparation formula had a slightly excellent performance when the samples were stored at room temperature or high temperature for a long time, while F2-1-F2-3 had a slightly excellent performance in the agitation and freeze-thawing experiments.
(11) ELISA: There was no significant difference in binding activity among all candidate formulas.

Summary: The comprehensive evaluation of the detection indicators of the 6 candidate formulas shows that the formula F2-4 (20 mM acetic acid-sodium acetate, pH 5.0; 0.04% (w/v) PS80; 8% (w/v) sucrose) is good in appearance, particle size, and purity and slightly superior in stability during long-term storage at room temperature or high temperature, as compared to other formulas. Thus, the F2-4 formula was selected for subsequent surfactant strength screening.

### Example 4. Surfactant Strength Screening

### 4.1 Study Protocol

(1) Based on the optimal pH/buffer system and excipient (20 mM acetic acid-sodium acetate pH 5.0; 8% (w/v) sucrose) obtained by screening in Examples 2 and 3, 3 different concentrations of surfactant (polysorbate 80, PS80) were designed, and the specific information of the candidate formula is shown in Table 28. (2) The stability of the PVRIG/TIGIT bispecific antibody LC-BsAb-002 (with a protein concentration of 50 mg/mL) in 3 candidate formulas was comprehensively investigated through a stability experiment, an agitation experiment, and a freeze-thawing experiment to select the optimal surfactant strength. The assessment indicators included appearance, pH, protein concentration, dynamic light scattering (DLS), cation exchange chromatography (CEX), SE-HPLC, CE-SDS (NR), and binding activity (ELISA-Binding). The investigation protocol is detailed in Table 33.

TABLE 33
Formulas and investigation protocol for surfactant strength screening

| | Formulation | | | | | | | | | | | | | | | |
|---|---|---|---|---|---|---|---|---|---|---|---|---|---|---|---|---|
| Formula | pH/buffer system/ | PS80 | | 2~8° C. | | | 25° C. | | | 40° C. | | | Agitation | | F/T | |
| No. | excipient | (w/v) | T0 | 4 W | 2 M | 6 M | 4 W | 2 M | 6 M | 1 W | 2 W | 4 W | 4 D | 7 D | 3C | 5C |
| F2-4-1 | 20 mM acetic | 0.02% | X, Y | X, Y | X, Y | X, Y | X, Y | X, Y | X, Y | X, Y | X, Y | X, Y | X | X, Y | X | X, Y |
| F2-4-2 | acid-sodium acetate, | 0.04% | | | | | | | | | | | | | | |
| F2-4-3 | pH 5.0; | 0.06% | | | | | | | | | | | | | | |
| | 8% sucrose | | | | | | | | | | | | | | | |

Note:
X = appearance, pH, protein concentration, DLS, iCIEF, SE-HPLC, CE-SDS (NR);
Y = ELISA-Binding, insoluble particle (MFI).

### 4.2 Screening Results

The major results of the surfactant strength screening are summarized in Tables 34-45 below:

TABLE 34
Appearance detection results for surfactant strength screening

| Formula | | 2~8° C. | | | 25° C. | | |
|---|---|---|---|---|---|---|---|
| No. | T0 | 4 W | 2 M | 6 M | 4 W | 2 M | 6 M |
| F2-4-1 | Colorless, | Colorless, | Colorless, | Colorless, | Colorless, | Colorless, | Colorless, |
| | slightly | slightly | slightly | slightly | slightly | slightly | slightly |
| | opalescent, | opalescent, | opalescent, | opalescent, | opalescent, | opalescent, | opalescent, |
| | no visible | no visible | no visible | no visible | no visible | no visible | no visible |
| | particles | particles | particles | particles | particles | particles | particles |
| F2-4-2 | Colorless, | Colorless, | Colorless, | Colorless, | Colorless, | Colorless, | Colorless, |
| | slightly | slightly | slightly | slightly | slightly | slightly | slightly |
| | opalescent, | opalescent, | opalescent, | opalescent, | opalescent, | opalescent, | opalescent, |
| | no visible | no visible | no visible | no visible | no visible | no visible | no visible |
| | particles | particles | particles | particles | particles | particles | particles |
| F2-4-3 | Colorless, | Colorless, | Colorless, | Colorless, | Colorless, | Colorless, | Colorless, |
| | slightly | slightly | slightly | slightly | slightly | slightly | slightly |
| | opalescent, | opalescent, | opalescent, | opalescent, | opalescent, | opalescent, | opalescent, |
| | no visible | no visible | no visible | no visible | no visible | no visible | no visible |
| | particles | particles | particles | particles | particles | particles | particles |
| Formula | 40° C. | | | Agitation | | F/T | |
| No. | 1 W | 2 W | 4 W | 4 D | 7 D | 3 C | 5 C |
| F2-4-1 | Colorless, | Colorless, | Colorless, | Colorless, | Colorless, | Colorless, | Colorless, |
| | slightly | slightly | slightly | slightly | slightly | slightly | slightly |
| | opalescent, | opalescent, | opalescent, | opalescent, | opalescent, | opalescent, | opalescent, |
| | no visible | no visible | no visible | no visible | no visible | no visible | no visible |
| | particles | particles | particles | particles | particles | particles | particles |
| F2-4-2 | Colorless, | Colorless, | Colorless, | Colorless, | Colorless, | Colorless, | Colorless, |
| | slightly | slightly | slightly | slightly | slightly | slightly | slightly |
| | opalescent, | opalescent, | opalescent, | opalescent, | opalescent, | opalescent | opalescent, |
| | no visible | no visible | no visible | no visible | no visible | no visible | no visible |
| | particles | particles | particles | particles | particles | particles | particles |
| F2-4-3 | Colorless, | Colorless, | Colorless, | Colorless, | Colorless, | Colorless, | Colorless, |
| | slightly | slightly | slightly | slightly | slightly | slightly | slightly |
| | opalescent, | opalescent, | opalescent, | opalescent, | opalescent, | opalescent, | opalescent, |
| | no visible | no visible | no visible | no visible | no visible | no visible | no visible |
| | particles | particles | particles | particles | particles | particles | particles |

TABLE 35
pH detection results for surfactant strength screening

| Formula | | 2~8° C. | | | 25° C. | | | 40° C. | | | Agitation | | F/T | |
|---|---|---|---|---|---|---|---|---|---|---|---|---|---|---|
| No. | T0 | 4 W | 2 M | 6 M | 4 W | 2 M | 6 M | 1 W | 2 W | 4 W | 4 D | 7 D | 3 C | 5 C |
| F2-4-1 | 5.2 | 5.2 | 5.2 | 5.2 | 5.2 | 5.2 | 5.2 | 5.2 | 5.2 | 5.2 | 5.2 | 5.2 | 5.2 | 5.2 |
| F2-4-2 | 5.2 | 5.2 | 5.2 | 5.2 | 5.2 | 5.2 | 5.2 | 5.2 | 5.2 | 5.2 | 5.2 | 5.2 | 5.2 | 5.2 |
| F2-4-3 | 5.2 | 5.2 | 5.2 | 5.2 | 5.2 | 5.2 | 5.2 | 5.2 | 5.2 | 5.2 | 5.2 | 5.2 | 5.2 | 5.2 |

TABLE 36
Protein concentration (mg/mL) detection results for surfactant strength screening

| Formula | | 2~8° C. | | | 25° C. | | | 40° C. | | | Agitation | | F/T | |
|---|---|---|---|---|---|---|---|---|---|---|---|---|---|---|
| No. | T0 | 4 W | 2 M | 6 M | 4 W | 2 M | 6 M | 1 W | 2 W | 4 W | 4 D | 7 D | 3 C | 5 C |
| F2-4-1 | 50.5 | 50.3 | 50.5 | 50.4 | 50.6 | 50.7 | 51.2 | 50.3 | 51.1 | 50.5 | 50.4 | 50.2 | 50.6 | 50.8 |
| F2-4-2 | 50.4 | 50.5 | 50.3 | 50.6 | 50.6 | 50.6 | 51.0 | 50.5 | 50.7 | 50.4 | 50.5 | 50.2 | 50.5 | 50.5 |
| F2-4-3 | 50.4 | 50.5 | 50.5 | 50.5 | 50.3 | 50.7 | 50.7 | 50.4 | 50.4 | 50.6 | 50.5 | 50.4 | 50.6 | 50.3 |

TABLE 37
DLS (particle size, nm) detection results for surfactant strength screening

| Formula | | 2~8° C. | | | 25° C. | | | 40° C. | | | Agitation | | F/T | |
|---|---|---|---|---|---|---|---|---|---|---|---|---|---|---|
| No. | T0 | 4 W | 2 M | 6 M | 4 W | 2 M | 6 M | 1 W | 2 W | 4 W | 4 D | 7 D | 3 C | 5 C |
| F2-4-1 | 9.9 | 10.0 | 10.2 | 10.5 | 9.6 | 10.1 | 10.6 | 9.2 | 10.5 | 10.6 | 9.9 | 9.7 | 9.8 | 9.8 |
| F2-4-2 | 9.8 | 9.9 | 10.1 | 10.6 | 9.7 | 9.9 | 10.7 | 9.2 | 10.4 | 10.4 | 9.6 | 9.8 | 10.2 | 9.6 |
| F2-4-3 | 10.1 | 9.9 | 10.5 | 10.3 | 9.9 | 9.9 | 10.7 | 9.3 | 10.4 | 10.2 | 9.5 | 9.9 | 10.1 | 9.9 |

TABLE 38
Insoluble particle (MFI, particles/mL) detection results for surfactant strength screening

| | | | 2~8° C. | | | | | | 25° C. | | | | | |
|---|---|---|---|---|---|---|---|---|---|---|---|---|---|---|
| Formula | T0 | | 4 W | | 2 M | | 6 M | | 4 W | | 2 M | | 6 M | |
| No. | ≥10 | ≥25 | ≥10 | ≥25 | ≥10 | ≥25 | ≥10 | ≥25 | ≥10 | ≥25 | ≥10 | ≥25 | ≥10 | ≥25 |
| F2-4-1 | 10 | 0 | 16 | 7 | 71 | 5 | 105 | 5 | 5 | 0 | 57 | 0 | 261 | 5 |
| F2-4-2 | 7 | 5 | 3 | 0 | 46 | 7 | 373 | 5 | 0 | 0 | 57 | 14 | 44 | 0 |
| F2-4-3 | 7 | 5 | 0 | 0 | 316 | 41 | 159 | 0 | 0 | 0 | 12 | 0 | 75 | 3 |

| | | | 40° C. | | | | | | Agitation | | F/T | |
|---|---|---|---|---|---|---|---|---|---|---|---|---|
| Formula | T0 | | 1 W | | 2 W | | 4 W | | 7 D | | 5 C | |
| No. | ≥10 | ≥25 | ≥10 | ≥25 | ≥10 | ≥25 | ≥10 | >25 | ≥10 | ≥25 | ≥10 | ≥25 |
| F2-4-1 | 10 | 0 | 3 | 0 | 78 | 3 | 10 | 0 | 3 | 3 | 3 | 0 |
| F2-4-2 | 7 | 5 | 3 | 0 | 25 | 0 | 0 | 0 | 12 | 3 | 21 | 3 |
| F2-4-3 | 7 | 5 | 5 | 0 | 7 | 3 | 5 | 0 | 7 | 0 | 3 | 3 |

TABLE 39
CEX (%) detection results of stability experiment at 2-8°
C. and 25° C. for surfactant strength screening

| | | 2~8° C. | | |
|---|---|---|---|---|
| Formula | T0 | 4 W | 2 M | 6 M |

| No. | MP | AP | BP | MP | AF | BP | MP | AP | BP | MP | AP | BP |
|---|---|---|---|---|---|---|---|---|---|---|---|---|
| F2-4-1 | 63.1 | 26.4 | 10.5 | 63.5 | 26.3 | 10.2 | 65 | 25.6 | 9.4 | 61.9 | 26.9 | 11.2 |
| F2-4-2 | 63.3 | 26.3 | 10.4 | 63.6 | 26.2 | 10.1 | 64.7 | 25.5 | 8.1 | 62 | 26.4 | 11.6 |
| F2-4-3 | 63.3 | 26.2 | 10.5 | 63.9 | 26 | 10.1 | 64.7 | 25.7 | 9.6 | 62.1 | 26.4 | 11.5 |

| | | | | 25° C. | | | | | | | | |
|---|---|---|---|---|---|---|---|---|---|---|---|---|
| Formula | T0 | | | 4 W | | | 2 M | | | 6 M | | |
| No. | MP | AP | BP | MP | AP | BP | MP | AP | BP | MP | AP | BP |
| F2-4-1 | 63.1 | 26.4 | 10.5 | 60.7 | 27.1 | 12.2 | 57.9 | 29.5 | 12.7 | 43.6 | 39.3 | 17.1 |
| F2-4-2 | 63.3 | 26.3 | 10.4 | 60.9 | 27.1 | 11.9 | 57.7 | 29.5 | 12.8 | 43.6 | 39.4 | 17 |
| F2-4-3 | 63.3 | 26.2 | 10.5 | 61 | 27.1 | 12 | 57.8 | 29.5 | 12.8 | 43.4 | 39.6 | 16.9 |

TABLE 40
CEX (%) detection results of stability experiment at 40° C., agitation
experiment, and freeze-thawing experiment for surfactant strength screening

| | | | | 40° C. | | | | | | | | |
|---|---|---|---|---|---|---|---|---|---|---|---|---|
| Formula | T0 | | | 1 W | | | 2 W | | | 4 W | | |
| No. | MP | AP | BP | MP | AP | BP | MP | AP | BP | MP | AP | BP |
| F2-4-1 | 63.1 | 26.4 | 10.5 | 56 | 30.5 | 13.6 | 50.3 | 34 | 15.6 | 39.9 | 41.6 | 18.5 |
| F2-4-2 | 63.3 | 26.3 | 10.4 | 56 | 30.4 | 13.5 | 50.6 | 34 | 15.4 | 40.3 | 41.8 | 17.9 |
| F2-4-3 | 63.3 | 26.2 | 10.5 | 56.3 | 30.4 | 13.4 | 50.5 | 34 | 15.5 | 39.7 | 41.9 | 18.4 |

| | | | | Agitation | | | | | | F/T | | | | | |
|---|---|---|---|---|---|---|---|---|---|---|---|---|---|---|---|
| Formula | T0 | | | 4 D | | | 7 D | | | 3 C | | | 5 C | | |
| No. | MP | AP | BP | MP | AP | BP | MP | AP | BP | MP | AP | BP | MF | AP | BP |
| F2-4-1 | 63.1 | 26.4 | 10.5 | 63.3 | 26.2 | 10.5 | 63 | 26.1 | 10.9 | 63.8 | 26.3 | 9.9 | 63.6 | 26.3 | 10 |
| F2-4-2 | 63.3 | 26.3 | 10.4 | 63.3 | 26.2 | 10.6 | 63 | 26.2 | 10.8 | 63.6 | 26.4 | 10 | 63.7 | 26.3 | 10 |
| F2-4-3 | 63.3 | 26.2 | 10.5 | 63.2 | 26.2 | 10.6 | 63.1 | 26.1 | 10.9 | 63.7 | 26.3 | 10 | 63.8 | 26.2 | 10 |

Note:
MP: main peak;
AP: acidic peak;
BP: basic peak

TABLE 41
SE-HPLC (%) detection results of stability experiment at 2-8° C. and 25° C. for surfactant strength screening

| | | | | 2~8° C. | | | | | | | | |
|---|---|---|---|---|---|---|---|---|---|---|---|---|
| Formula | T0 | | | 4 W | | | 2 M | | | 6 M | | |
| No. | MP | HMW | LMW | MP | HMW | LMW | MP | HMW | LMW | MP | HMW | LMW |
| F2-4-1 | 99.1 | 0.9 | ND | 98.8 | 1.2 | ND | 98.8 | 1.2 | ND | 98.6 | 1.4 | ND |
| F2-4-2 | 99.1 | 0.9 | ND | 98.6 | 1.4 | ND | 98.8 | 1.2 | ND | 98.6 | 1.4 | ND |
| F2-4-3 | 99.1 | 0.9 | ND | 98.7 | 1.3 | ND | 98.8 | 1.2 | ND | 98.6 | 1.4 | ND |
| | | | | 25° C. | | | | | | | | |
| Formula | T0 | | | 4 W | | | 2 M | | | 6 M | | |
| No. | MP | HMW | LMW | MP | HMW | LMW | MP | HMW | LMW | MP | HMW | LMW |
| F2-4-1 | 99.1 | 0.9 | ND | 98.3 | 1.6 | 0.2 | 96.3 | 1.7 | 2 | 94.7 | 2.5 | 2.8 |
| F2-4-2 | 99.1 | 0.9 | ND | 98.2 | 1.6 | 0.2 | 96.4 | 1.7 | 2 | 94.6 | 2.6 | 2.8 |
| F2-4-3 | 99.1 | 0.9 | ND | 98.2 | 1.6 | 0.2 | 96.3 | 1.7 | 2 | 94.7 | 2.4 | 2.8 |

TABLE 42
SE-HPLC (%) detection results of stability experiment at 40° C., agitation
experiment, and freeze-thawing experiment for surfactant strength screening

| | | | | 40° C. | | | | | | | | |
|---|---|---|---|---|---|---|---|---|---|---|---|---|
| Formula | T0 | | | 1 W | | | 2 W | | | 4 W | | |
| No. | MP | HMW | LMW | MP | HMW | LMW | MP | HMW | LMW | MP | HMW | LMW |
| F2-4-1 | 99.1 | 0.9 | ND | 96.7 | 1.5 | 1.7 | 95.6 | 2.2 | 2.2 | 92.6 | 3.6 | 3.8 |
| F2-4-2 | 99.1 | 0.9 | ND | 96.9 | 1.4 | 1.7 | 95.4 | 2.2 | 2.4 | 92.5 | 3.6 | 3.9 |
| F2-4-3 | 99.1 | 0.9 | ND | 96.7 | 1.5 | 1.7 | 95.4 | 2.2 | 2.3 | 92.7 | 3.5 | 3.8 |

| | | | | Agitation | | | | | | F/T | | | | | |
|---|---|---|---|---|---|---|---|---|---|---|---|---|---|---|---|
| Formula | T0 | | | 4 D | | | 7 D | | | 3 C | | | 5 C | | |
| No. | MP | HMW | LMW | MP | HMW | LMW | MP | HMW | LMW | MP | HMW | LMW | MP | HMW | LMW |
| F2-4-1 | 99.1 | 0.9 | ND | 99 | 1 | ND | 98.7 | 1.1 | 0.1 | 99 | 1 | ND | 98.9 | 1.1 | ND |
| F2-4-2 | 99.1 | 0.9 | ND | 98.9 | 1.1 | ND | 98.9 | 1 | 0.1 | 99.1 | 0.9 | ND | 99 | 1 | ND |
| F2-4-3 | 99.1 | 0.9 | ND | 99 | 1 | ND | 98.9 | 1.1 | 0.1 | 99 | 1 | ND | 99 | 1 | ND |

Note:
MP: main peak;
HMW: high molecular weight, high polymer;
LMW: low molecular weight, oligomer;
ND: not detected

TABLE 43
CE-SDS-NR (%) detection results of stability experiment at 2-8°
C. and 25° C. for surfactant strength screening

| | | | | 2~8° C. | | | | | | | | |
|---|---|---|---|---|---|---|---|---|---|---|---|---|
| Formula | T0 | | | 4 W | | | 2 M | | | 6 M | | |
| No. | MP | HMW | LMW | MP | HMW | LMW | MP | HMW | LMW | MP | HMW | LMW |
| F2-4-1 | 96.9 | 1 | 2 | 97.8 | 0.1 | 2.1 | 98.1 | 0.1 | 1.8 | 97.4 | 0.5 | 2.1 |
| F2-4-2 | 97.9 | 0 | 2.1 | 97.9 | 0.1 | 2 | 98.1 | 0.1 | 1.8 | 97.0 | 0.6 | 2.5 |
| F2-4-3 | 97.2 | 0.7 | 2.1 | 98 | 0.1 | 2 | 98 | 0.1 | 1.9 | 96.7 | 0.7 | 2.6 |
| | | | | 25° C. | | | | | | | | |
| Formula | T0 | | | 4 W | | | 2 M | | | 6 M | | |
| No. | MP | HMW | LMW | MP | HMW | LMW | MP | HMW | LMW | MP | HMW | LMW |
| F2-4-1 | 96.9 | 1 | 2 | 96.8 | 0.6 | 2.6 | 96.8 | 0.1 | 3.1 | 92.7 | 0.8 | 6.5 |
| F2-4-2 | 97.9 | 0 | 2.1 | 97.2 | 0.5 | 2.4 | 96.9 | 0.1 | 3.1 | 93.0 | 0.8 | 6.2 |
| F2-4-3 | 97.2 | 0.7 | 2.1 | 97.4 | 0 | 2.7 | 96.8 | 0.1 | 3 | 92.5 | 1.1 | 6.4 |

TABLE 44
CE-SDS-NR (%) detection results of stability experiment at 40° C., agitation
experiment, and freeze-thawing experiment for surfactant strength screening

| | | | | 40° C. | | | | | | | | |
|---|---|---|---|---|---|---|---|---|---|---|---|---|
| Formula | T0 | | | 1 W | | | 2 W | | | 4 W | | |
| No. | MP | HMW | LMW | MP | HMW | LMW | MP | HMW | LMW | MP | HMW | LMW |
| F2-4-1 | 96.9 | 1 | 2 | 96.6 | 0.1 | 3.3 | 95.3 | 0.4 | 4.2 | 93.3 | 0.6 | 6.2 |
| F2-4-2 | 97.9 | 0 | 2.1 | 96.1 | 0.1 | 3.8 | 95.7 | 0.2 | 4.2 | 93.7 | 0.2 | 6 |
| F2-4-3 | 97.2 | 0.7 | 2.1 | 96.4 | 0.1 | 3.4 | 95.5 | 0.2 | 4.2 | 93.5 | 0.4 | 6.1 |

| | | | | Agitation | | | | | | F/T | | | | | |
|---|---|---|---|---|---|---|---|---|---|---|---|---|---|---|---|
| Formula | T0 | | | 4 D | | | 7 D | | | 3 C | | | 5 C | | |
| No. | MP | HMW | LMW | MP | HMW | LMW | MP | HMW | LMW | MP | HMW | LMW | MP | HMW | LMW |
| F2-4-1 | 96.9 | 1 | 2 | 97.1 | 0.7 | 2.2 | 95.9 | 1.9 | 2.1 | 97.7 | 0.4 | 1.8 | 96.8 | 1.1 | 2.2 |
| F2-4-2 | 97.9 | 0 | 2.1 | 97.5 | 0.5 | 2 | 95.5 | 2.3 | 2.1 | 97.1 | 0.8 | 2.1 | 97 | 0.8 | 2 |
| F2-4-3 | 97.2 | 0.7 | 2.1 | 97.2 | 0.6 | 2.1 | 97.2 | 0.5 | 2.2 | 96.7 | 0.9 | 2.5 | 97.1 | 0.9 | 2 |

Note:
MP: main peak;
HMW: high molecular weight, high polymer;
LMW: low molecular weight, oligomer

TABLE 45
ELISA-Binding (%) detection results for surfactant strength screening

| Formula | | 2~8° C. | | | 25° C. | | | 40° C. | | | Agitation | F/T |
|---|---|---|---|---|---|---|---|---|---|---|---|---|
| No. | T0 | 4 W | 2 M | 6 M | 4 W | 2 M | 6 M | 1 W | 2 W | 4 W | 7 D | 5 C |
| F2-4-1 | 100.52 | 97.97 | 96.99 | 87 | 86.4 | 91.16 | 75 | 87.18 | 95.78 | 84.34 | 90.79 | 85.96 |
| F2-4-2 | 95.1 | 95.96 | 94.85 | 89 | 86.65 | 85.98 | 76 | 90.45 | 95.34 | 87.25 | 95.74 | 80.17 |
| F2-4-3 | 94.18 | 94.97 | 100.59 | 83 | 99.3 | 91.71 | 90 | 81.62 | 92.93 | 84.26 | 92.08 | 95.93 |

### 4.3 Analysis of Results

(1) Appearance: The appearance of all candidate formulas was consistent, with no apparent change throughout the investigation.
(2) pH value: The pH of all candidate formulas did not change significantly throughout the investigation.
(3) Protein concentration: The protein concentration of all candidate formulas did not change significantly throughout the investigation.
(4) Insoluble particle: According to the quality standard (the number of particles (≥10 μm)≤6000 particles, and the number of particles (≥25 μm)≤600 particles), the insoluble particles of all candidate formulas showed no significant abnormality throughout the investigation and met the quality standard, where F2-4-2 had a slightly excellent performance.
(5) DLS: There was no significant difference in particle size among all candidate formulas.
(6) CEX: There was no significant difference in charge isomer main peak among all candidate formulas.
(7) SE-HPLC: There was no significant difference in monomer purity among all candidate formulas.
(8) CE-SDS (NR): There was no significant difference in protein purity among all candidate formulas.
(9) ELISA: There was no significant difference in binding activity among all candidate formulas.

Summary: The comprehensive evaluation of the detection indicators of the 3 candidate formulas shows that there was no significant difference in the effect on protein stability by the concentrations of 0.02-0.06% (w/v) PS80. Thus, the concentration of 0.04% (w/v) PS80 used in the excipient screening study continued to be used, and the F2-4-2 formula was selected for subsequent formula stability confirmation study.

### Example 5. Formula Stability Confirmation

### 5.1 Study Protocol

(1) The formula (20 mM acetic acid-sodium acetate, pH 5.0; 8% (w/v) sucrose: 0.04% (w/v) PS80) was selected for the formula stability confirmation study. (2) A 10-mL vial, a 10-mm rubber stopper, and a 10-mm aluminum plastic cap were selected as packaging materials. (3) Specification: 50 mg/mL PVRIG/TIGIT bispecific antibody LC-BsAb-002; loading: 6 mL/300 mg. The assessment indicators included appearance, pH, protein concentration, dynamic light scattering (DLS), CEX, SE-HPLC, CE-SDS (NR&R), and binding activity (ELISA-Binding). The investigation protocol is detailed in Table 46.

TABLE 46
Investigation protocol for formula stability confirmation

| Condition | | 2~8° C. | | | 25° C. | | | 40° C. | |
|---|---|---|---|---|---|---|---|---|---|
| of standing | T0 | 4 W | 3 M | 6 M | 4 W | 3 M | 6 M | 2 W | 4 W |
| Upright (U) | X | X | X | X | X | X | X | X | X |
| Inverted (I) | | | | | | | | | |

Note:
X = appearance, pH, protein concentration, DLS, iCIEF, SE-HPLC, CE-SDS (NR), ELISA-Binding, insoluble particle (MFI).

### 5.2 Investigation Results

The major results of the formula stability confirmation are summarized in Tables 47-48:

TABLE 47
Formula stability confirmation results

| Sampling | | Condition | | | Concen- | Particle | Binding |
|---|---|---|---|---|---|---|---|
| point | | of standing | Appearance | pH | tration | size | activity (%) |
| T0 | | | Colorless, slightly | 5.1 | 49.8 | 9.6 | 90.68 |
| | | | opalescent, no visible | | | | |
| | | | particles | | | | |
| 2~8° C. | 4 W | U | Colorless, slightly | 5.1 | 50.2 | 9.4 | 92.98 |
| | | | opalescent, no visible | | | | |
| | | | particles | | | | |
| | | I | Colorless, slightly | 5.1 | 50.4 | 9.2 | 89.65 |
| | | | opalescent, no visible | | | | |
| | | | particles | | | | |
| | 3 M | U | Colorless, slightly | 5.1 | 49.9 | 9.2 | 79 |
| | | | opalescent, no visible | | | | |
| | | | particles | | | | |
| | | I | Colorless, slightly | 5.1 | 49.9 | 8.9 | 82 |
| | | | opalescent, no visible | | | | |
| | | | particles | | | | |
| | 6 M | U | Colorless, slightly | 5.2 | 50.0 | 9.1 | 96 |
| | | | opalescent, no visible | | | | |
| | | | particles | | | | |
| | | I | Colorless, slightly | 5.2 | 50.0 | 9.2 | 93 |
| | | | opalescent, no visible | | | | |
| | | | particles | | | | |
| 25° C. | 4 W | U | Colorless, slightly | 5.1 | 50.2 | 9.1 | 80.83 |
| | | | opalescent, no visible | | | | |
| | | | particles | | | | |
| | | I | Colorless, slightly | 5.1 | 50.4 | 8.8 | 86.72 |
| | | | opalescent, no visible | | | | |
| | | | particles | | | | |
| | 3 M | U | Colorless, slightly | 5.1 | 50.1 | 9.6 | 85 |
| | | | opalescent, no visible | | | | |
| | | | particles | | | | |
| | | I | Colorless, slightly | 5.1 | 50.0 | 9.6 | 85 |
| | | | opalescent, no visible | | | | |
| | | | particles | | | | |
| | 6 M | U | Colorless, slightly | 5.2 | 50.0 | 10.9 | 80 |
| | | | opalescent, no visible | | | | |
| | | | particles | | | | |
| | | I | Colorless, slightly | 5.2 | 49.8 | 9.8 | 80 |
| | | | opalescent, no visible | | | | |
| | | | particles | | | | |
| 40° C. | 2 W | U | Colorless, slightly | 5.1 | 50.1 | 9.2 | 87.97 |
| | | | opalescent, no visible | | | | |
| | | | particles | | | | |
| | | I | Colorless, slightly | 5.1 | 49.7 | 9.2 | 90.82 |
| | | | opalescent, no visible | | | | |
| | | | particles | | | | |
| | 4 W | U | Colorless, slightly | 5.1 | 49.9 | 10.2 | 74.14 |
| | | | opalescent, no visible | | | | |
| | | | particles | | | | |
| | | I | Colorless, slightly | 5.1 | 50.1 | 10.6 | 79.42 |
| | | | opalescent, no visible | | | | |
| | | | particles | | | | |

TABLE 48
Formula stability confirmation results

| | | | Insoluble particle | | | | | | | | | | | | |
|---|---|---|---|---|---|---|---|---|---|---|---|---|---|---|---|
| Sampling | | Condition | (MFI, particles/mL) | | CEX (%) | | | SE-HPLC (%) | | | CE-SDS-NR (%) | | | CE-SDS-R (%) | |
| point | | of standing | ≥10 | ≥25 | MP | AP | BP | MP | HMW | LMW | MP | HMW | LMW | LC + HC | NGHC |
| T0 | | | 3 | 0 | 63.3 | 27.1 | 9.6 | 98.8 | 1.2 | ND | 98.3 | 0.3 | 1.3 | 98.0 | 2.0 |
| 2~8° C. | 4 W | U | 25 | 5 | 65.1 | 26.3 | 8.7 | 98.6 | 1.4 | ND | 97.6 | 0.2 | 2.2 | 97.8 | 2.2 |
| | | I | 7 | 0 | 64.8 | 26.4 | 8.9 | 98.7 | 1.3 | ND | 96.5 | 1.4 | 2.1 | 97.2 | 2.8 |
| | 3 M | U | 23 | 3 | 63.5 | 26.2 | 10.3 | 98.2 | 1.8 | ND | 97.7 | 0.9 | 1.4 | 97.8 | 2.2 |
| | | I | 12 | 3 | 63.4 | 26.3 | 10.3 | 98.3 | 1.7 | ND | 97.6 | 0.8 | 1.6 | 97.6 | 2.4 |
| | 6 M | U | 11 | 3 | 62.7 | 25.7 | 11.6 | 98.1 | 1.9 | ND | 98.0 | 0.4 | 1.6 | 97.5 | 0.8 |
| | | I | 8 | 0 | 62.9 | 25.7 | 11.4 | 98 | 2 | ND | 98.2 | 0.3 | 1.6 | 97.3 | 0.9 |
| 25° C. | 4 W | U | 14 | 0 | 61.6 | 27.3 | 11.1 | 97.7 | 2.1 | 0.2 | 97.0 | 1.2 | 1.9 | 96.2 | 3.8 |
| | | I | 3 | 0 | 62 | 27.3 | 10.8 | 97.8 | 2.1 | 0.1 | 96.5 | 1.4 | 2.0 | 97.4 | 2.6 |
| | 3 M | U | 100 | 0 | 52.9 | 31.5 | 15.5 | 95.9 | 2.7 | 1.4 | 96.3 | 1.0 | 2.8 | 95.3 | 4.7 |
| | | I | 94 | 10 | 52.3 | 31.7 | 16 | 95.5 | 2.7 | 1.7 | 96.7 | 0.8 | 2.6 | 95.4 | 4.6 |
| | 6 M | U | 11 | 0 | 45.3 | 36 | 18.6 | 95.3 | 2.9 | 1.8 | 94.2 | 1.2 | 4.7 | 94.2 | 1.0 |
| | | I | 16 | 0 | 43.9 | 36.3 | 19.7 | 94.7 | 3.3 | 2 | 94.3 | 1.2 | 4.5 | 93.3 | 1.0 |
| 40° C. | 2 W | U | 16 | 3 | 50.5 | 34.3 | 15.1 | 94.9 | 3 | 2.1 | 95.3 | 1.3 | 3.4 | 97.2 | 2.8 |
| | | I | 19 | 0 | 51.4 | 34.3 | 14.1 | 95 | 3 | 2 | 95.5 | 1.2 | 3.3 | 96.5 | 3.5 |
| | 4 W | U | 30 | 3 | 40.5 | 41.1 | 18.4 | 92.7 | 4.1 | 3.2 | 93.4 | 1.6 | 5.0 | 93.4 | 6.6 |
| | | I | 12 | 0 | 40.3 | 40.9 | 18.7 | 92.6 | 4.1 | 3.3 | 94.2 | 1.0 | 4.8 | 94.0 | 6.0 |

### 5.3 Analysis of Results

(1) Appearance: The appearance of the formula did not change significantly throughout the investigation.
(2) pH value: The pH of the formula did not change significantly throughout the investigation.
(3) Protein concentration: The protein concentration of the formula did not change significantly throughout the investigation.
(4) Insoluble particle: The insoluble particles of the formula showed no significant abnormality throughout the investigation and met the quality standard.
(5) DLS: The particle size of the formula did not change significantly throughout the investigation.
(6) CEX: The charge isomer main peak change of the formula met the quality standard when the sample was stored at low, room, and high temperatures.
(7) SE-HPLC: The monomer purity of the formula showed good stability when the sample was stored at low, room and high temperatures.
(8) CE-SDS (NR&R): The protein purity of the formula changed slightly and showed good stability when the sample was stored at low, room, and high temperatures.
(9) ELISA-Binding: The binding activity of the formula did not change significantly throughout the investigation.

Summary: All detection indicators of the formula meet the quality standard under the investigation conditions of 2-8° C., 25° C., and 40° C., the formula shows good stability, and the sample shows no significant difference when being placed upright and inverted.

In conclusion, after pH/buffer system screening, excipient screening, surfactant strength screening, and formula stability confirmation study, the final formula was determined to be 50 mg/mL PVRIG/TIGIT bispecific antibody; 20 mM acetic acid-sodium acetate, pH 5.0; 8% (w/v) sucrose; and 0.04% (w/v) PS80.

Although the present disclosure finally selected 20 mM acetic acid-sodium acetate, pH 5.0; 8% (w/v) sucrose; 0.04% (w/v) PS80 for the formula, it does not mean that only this formula can achieve the effect of stabilizing the antibody. As with the data of the previous examples, stable storage of the antibody can also be achieved using a histidine buffer system, as well as using sorbitol and trehalose as an excipient.

1. A pharmaceutical composition, comprising:
(i) a bispecific antibody or an antigen-binding fragment thereof comprising a binding domain that specifically binds to PVRIG and TIGIT,
(ii) a buffer,
(iii) a non-reducing sugar, and
(iv) a non-ionic surfactant,
wherein the bispecific antibody or the antigen-binding fragment thereof comprises:
(a) a first antigen-binding moiety comprising a heavy chain variable region (VH) and a light chain variable region (VL), wherein the VH and VL form an anti-TIGIT antigen-binding domain, wherein the TIGIT VH comprises HCDR1, HCDR2, and HCDR3 set forth in the following sequences:
(1) HCDR1, HCDR2, and HCDR3 set forth in SEQ ID NOs: 21, 22, and 23, respectively; or
(2) HCDR1, HCDR2, and HCDR3 set forth in SEQ ID NOs: 24, 25, and 26, respectively;
the TIGIT VL comprises LCDR1, LCDR2, and LCDR3 set forth in the following sequences:
(1) LCDR1, LCDR2, and LCDR3 set forth in SEQ ID NOs: 27, 28, and 29, respectively; or
(2) LCDR1, LCDR2, and LCDR3 set forth in SEQ ID NOs: 30, 31, and 32, respectively;
(b) a second antigen-binding moiety comprising a VHH that specifically binds to PVRIG, wherein the VHH comprises CDR1, CDR2, and CDR3 set forth in the following sequences:
(1) CDR1, CDR2, and CDR3 set forth in SEQ ID NOs: 15, 16, and 17, respectively; or
(2) CDR1, CDR2, and CDR3 set forth in SEQ ID NOs: 18, 19, and 20, respectively.

2. The pharmaceutical composition according to claim 1, wherein,
the TIGIT VH comprises the amino acid sequence set forth in any one of SEQ ID NO: 12 or 14;
the TIGIT VL comprises the amino acid sequence set forth in any one of SEQ ID NO: 11 or 13;
the VHH that specifically binds to PVRIG comprises the amino acid sequence set forth in any one of SEQ ID NO: 9 or 10.

3. The pharmaceutical composition according to claim 1,
wherein,
the bispecific antibody or the antigen-binding fragment thereof is LC-BsAb-002, LC-BsAb-006, LC-BsAb-009, or LC-BsAb-010;
the LC-BsAb-002 comprises:
(1) an LC with the amino acid sequence set forth in SEQ ID NO: 1; and
(2) an HC with the amino acid sequence set forth in SEQ ID NO: 2;
the LC-BsAb-006 comprises:
(3) an LC with the amino acid sequence set forth in SEQ ID NO: 3; and
(4) an HC with the amino acid sequence set forth in SEQ ID NO: 4;
the LC-BsAb-009 comprises:
(1) an LC with the amino acid sequence set forth in SEQ ID NO: 5; and
(2) an HC with the amino acid sequence set forth in SEQ ID NO: 6;
the LC-BsAb-010 comprises:
(3) an LC with the amino acid sequence set forth in SEQ ID NO: 7; and
(4) an HC with the amino acid sequence set forth in SEQ ID NO: 8.

4. The pharmaceutical composition according to claim 1, wherein the PVRIG/TIGIT bispecific antibody or the antigen-binding fragment thereof is at a concentration of about 10 mg/mL to about 200 mg/mL, preferably, about 20 mg/mL to about 100 mg/mL, about 20 mg/mL to about 80 mg/mL, or about 30 mg/mL to about 60 mg/mL; most preferably, 50 mg/mL.

5. The pharmaceutical composition according to claim 1, wherein the buffer is selected from an acetic acid-sodium acetate buffer or a histidine-histidine hydrochloride buffer; preferably, the buffer is at a concentration of about 10 mM to about 80 mM, about 15 mM to about 70 mM, about 20 mM to about 60 mM, or about 20 mM to about 30 mM; most preferably, a 20 mM acetic acid-sodium acetate buffer.

6. The pharmaceutical composition according to claim 1, wherein the non-reducing sugar is selected from sucrose, sorbitol, or trehalose; preferably, the non-reducing sugar is at a concentration of about 6% to about 9% (w/v), about 7% to about 9% (w/v), or about 7% to about 8% (w/v); most preferably, 8% w/v sucrose.

7. The pharmaceutical composition according to claim 1, wherein the non-ionic surfactant is polysorbate 80; preferably, the non-ionic surfactant is at a concentration of about 0.01% to about 0.10% (w/v), or about 0.02% to about 0.06% (w/v); most preferably, 0.04% (w/v) polysorbate 80.

8. The pharmaceutical composition according to claim 1, wherein the pharmaceutical composition is an injection, preferably a subcutaneous injection or an intravenous injection.

9. The pharmaceutical composition according to claim 1, comprising:
(i) 10 mg/mL to 200 mg/mL of the PVRIG/TIGIT bispecific antibody or the antigen-binding fragment thereof;
(ii) 10 mM to 100 mM acetic acid-sodium acetate buffer;
(iii) 6% to 10% (w/v) sucrose; and
(iv) 0.01% to 0.10% (w/v) polysorbate 80.

10. The pharmaceutical composition according to claim 9, comprising 50 mg/mL of the PVRIG/TIGIT bispecific antibody or the antigen-binding fragment thereof, 20 mM acetic acid-sodium acetate buffer, 8% w/v sucrose, and 0.02% (w/v) polysorbate 80.

11. The pharmaceutical composition according to claim 9, comprising 50 mg/mL of the PVRIG/TIGIT bispecific antibody or the antigen-binding fragment thereof, 20 mM acetic acid-sodium acetate buffer, 8% w/v sucrose, and 0.04% (w/v) polysorbate 80.

12. The pharmaceutical composition according to claim 9, comprising 50 mg/mL of the PVRIG/TIGIT bispecific antibody or the antigen-binding fragment thereof, 20 mM acetic acid-sodium acetate buffer, 8% w/v sucrose, and 0.06% (w/v) polysorbate 80.

13. The pharmaceutical composition according to claim 1, wherein the pH of the pharmaceutical composition is 4.5-5.5.

14. The pharmaceutical composition according to claim 13, wherein the pH of the pharmaceutical composition is 5.0-5.2.

15. (canceled)

16. (canceled)

17. (canceled)

18. A method for treating a cancer or an infectious disease, comprising administering to a patient in need thereof an effective amount of the pharmaceutical composition according to claim 1, wherein the cancer is selected from a solid tumor and a hematologic tumor.

19. A method for treating a cancer or an infectious disease, comprising administering to a patient in need thereof an effective amount of the pharmaceutical composition according to claim 1 in combination with an additional therapeutic agent, radiation therapy, or surgery, wherein the additional therapeutic agent is selected from chemotherapy, an oncolytic drug, a cytotoxic agent, a cytokine, an immunostimulatory antibody, an immunomodulatory drug, an activator of a costimulatory molecule, an inhibitor of an inhibitory molecule, a vaccine, or cellular immunotherapy, wherein the cancer is selected from a solid tumor and a hematologic tumor.


SEQUENCE LISTING (32 sequences)

SEQ ID NO: 1 - protein, 214 aa, synthetic construct
DIVMTQSPDSLAVSLGERATINCKASQNVRTAVAWYQQKPGQSPKLMIYSASYRYTGVPDRFSGSGSGTDFTLTISSLQAEDVAVYYCQQYYTTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 597 aa, synthetic construct
EVQLVESGGGLVQPGGSLRLSCAASGFTFSYYDMSWVRQAPGKGLEWVSTINSDGGRTSYVDSVKGRFTISRDNSKNTLYLQMNSLRAEDTAVYYCVEGDPHNFGLESLSLRDFGSWGQGTMVTVSSGGGGSGGGGSGGGGSGGGGSEVQLQESGPGLVKPSETLSLTCAVSGYSITSDSWNWIRQPPGKKLEYIGYISYSGNTYYNPSLKSRVTISRDTSKNQFSLKLSSVTAADTAVYYCARLDFSNYGGAVDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, synthetic construct
DIVMTQSPDSLAVSLGERATINCKASQHVSNAVAWYQHKPGQSPKLLIYSASYRYTGVPDRFSGSGSGTDFTLTISSLQAEDVAVYYCQQHYNTPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 594 aa, synthetic construct
EVQLVESGGGLVQPGGSLRLSCAASGFTFSYYDMSWVRQAPGKGLEWVSTINSDGGRTSYVDSVKGRFTISRDNSKNTLYLQMNSLRAEDTAVYYCVEGDPHNFGLESLSLRDFGSWGQGTMVTVSSGGGGSGGGGSGGGGSGGGGSEVQLVQSGAEVKKPGASVKVSCKASGYAFTNYLIEWVRQAPGQRLEWMGVINPGSGGTNYKEKFKGRVTITADKSSSTAYMELSSLRSEDTAVYYCARGEYFFFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, synthetic construct; identical to SEQ ID NO: 1

SEQ ID NO: 6 - protein, 598 aa, synthetic construct
EVQLVESGGGLVQPGGSLRLSCAASGRTFDRHTMSWFRQAPGKEREFVATASRIPGDTYYVDSVKGRFTISRDNAKNSLYLQMNSLRAEDTAVYYCAATSAYCSEVDCYEKGSWYDNWGQGTMVTVSSGGGGSGGGGSGGGGSGGGGSEVQLQESGPGLVKPSETLSLTCAVSGYSITSDSWNWIRQPPGKKLEYIGYISYSGNTYYNPSLKSRVTISRDTSKNQFSLKLSSVTAADTAVYYCARLDFSNYGGAVDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, synthetic construct; identical to SEQ ID NO: 3

SEQ ID NO: 8 - protein, 595 aa, synthetic construct
EVQLVESGGGLVQPGGSLRLSCAASGRTFDRHTMSWFRQAPGKEREFVATASRIPGDTYYVDSVKGRFTISRDNAKNSLYLQMNSLRAEDTAVYYCAATSAYCSEVDCYEKGSWYDNWGQGTMVTVSSGGGGSGGGGSGGGGSGGGGSEVQLVQSGAEVKKPGASVKVSCKASGYAFTNYLIEWVRQAPGQRLEWMGVINPGSGGTNYKEKFKGRVTITADKSSSTAYMELSSLRSEDTAVYYCARGEYFFFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 127 aa, synthetic construct
EVQLVESGGGLVQPGGSLRLSCAASGFTFSYYDMSWVRQAPGKGLEWVSTINSDGGRTSYVDSVKGRFTISRDNSKNTLYLQMNSLRAEDTAVYYCVEGDPHNFGLESLSLRDFGSWGQGTMVTVSS

SEQ ID NO: 10 - protein, 128 aa, synthetic construct
EVQLVESGGGLVQPGGSLRLSCAASGRTFDRHTMSWFRQAPGKEREFVATASRIPGDTYYVDSVKGRFTISRDNAKNSLYLQMNSLRAEDTAVYYCAATSAYCSEVDCYEKGSWYDNWGQGTMVTVSS

SEQ ID NO: 11 - protein, 107 aa, synthetic construct
DIVMTQSPDSLAVSLGERATINCKASQNVRTAVAWYQQKPGQSPKLMIYSASYRYTGVPDRFSGSGSGTDFTLTISSLQAEDVAVYYCQQYYTTPWTFGGGTKVEIK

SEQ ID NO: 12 - protein, 120 aa, synthetic construct
EVQLQESGPGLVKPSETLSLTCAVSGYSITSDSWNWIRQPPGKKLEYIGYISYSGNTYYNPSLKSRVTISRDTSKNQFSLKLSSVTAADTAVYYCARLDFSNYGGAVDYWGQGTTVTVSS

SEQ ID NO: 13 - protein, 107 aa, synthetic construct
DIVMTQSPDSLAVSLGERATINCKASQHVSNAVAWYQHKPGQSPKLLIYSASYRYTGVPDRFSGSGSGTDFTLTISSLQAEDVAVYYCQQHYNTPHTFGGGTKVEIK

SEQ ID NO: 14 - protein, 117 aa, synthetic construct
EVQLVQSGAEVKKPGASVKVSCKASGYAFTNYLIEWVRQAPGQRLEWMGVINPGSGGTNYKEKFKGRVTITADKSSSTAYMELSSLRSEDTAVYYCARGEYFFFDYWGQGTTVTVSS

SEQ ID NO: 15 - protein, 5 aa, synthetic construct
YYDMS

SEQ ID NO: 16 - protein, 17 aa, synthetic construct
TINSNGGRTSYVDSVKG

SEQ ID NO: 17 - protein, 18 aa, synthetic construct
GDPHNFGLENLSLRDFGS

SEQ ID NO: 18 - protein, 5 aa, synthetic construct
RHTMS

SEQ ID NO: 19 - protein, 17 aa, synthetic construct
TASRIPGDTYYVDSVKG

SEQ ID NO: 20 - protein, 19 aa, synthetic construct
TSAYCSEVDCYEKGSWYDN

SEQ ID NO: 21 - protein, 5 aa, synthetic construct
SDSWN

SEQ ID NO: 22 - protein, 16 aa, synthetic construct
YISYSGNTYYNPSLKS

SEQ ID NO: 23 - protein, 12 aa, synthetic construct
LDFSNYGGAVDY

SEQ ID NO: 24 - protein, 5 aa, synthetic construct
NYLIE

SEQ ID NO: 25 - protein, 17 aa, synthetic construct
VINPGSGGTNYKEKFKG

SEQ ID NO: 26 - protein, 8 aa, synthetic construct
GEYFFFDY

SEQ ID NO: 27 - protein, 11 aa, synthetic construct
KASQNVRTAVA

SEQ ID NO: 28 - protein, 7 aa, synthetic construct
SASYRYT

SEQ ID NO: 29 - protein, 9 aa, synthetic construct
QQYYTTPWT

SEQ ID NO: 30 - protein, 11 aa, synthetic construct
KASQHVSNAVA

SEQ ID NO: 31 - protein, 7 aa, synthetic construct
SASYRYT

SEQ ID NO: 32 - protein, 9 aa, synthetic construct
QQHYNTPHT